(19) United States
(12) Patent Application Publication
(10) Pub. No.: US 2023/0398232 A1
(43) Pub. Date: Dec. 14, 2023

(54) Composition and Use of Alternatively Formatted Anti-Mesothelin Antibodies for the Treatment of Cancer

(71) Applicant: Navrogen, Inc., Glen Mills, PA (US)

(72) Inventors: Luigi Grasso, Bryn Mawr, PA (US); Nicholas C. Nicolaides, Glen Mills, PA (US); James Bradford Kline, Morgantown, PA (US)

(21) Appl. No.: 18/026,518

(22) PCT Filed: Sep. 28, 2021
(86) PCT No.: PCT/US2021/052286

§ 371 Date: Mar. 15, 2023

Related U.S. Application Data
(60) Provisional application No. 63/084,542, filed on Sep. 28, 2020.

(51) Int. Cl. A61K 47/68; C07K 16/30; A61P 35/00; C07K 16/28

(52) U.S. Cl. CPC A61K 47/6863; A61K 47/6857; A61K 47/6869; A61K 47/6859; A61K 47/6849; C07K 16/30; A61K 47/6809; A61K 47/6803; A61K 47/6889; A61P 35/00; C07K 16/2809; A61K 2039/505

(57) ABSTRACT

Tumors utilize multiple mechanisms to avoid the host's anti-tumor immune response. Humoral immunosuppression is one of the mechanisms. Tumors produce circulating factors that can suppress antibody or complement-mediated immune responses to enhance their own survival. Mesothelin is a cell surface protein overexpressed by several cancer types that are associated with tumors exhibiting immunosuppressed microenvironments. Anti-mesothelin antibodies are often subject to such immunosuppressive microenvironments. Alternatively formatted anti-mesothelin antibodies, however, are effective in killing mesothelin-expressing cancers irrespective of tumor microenvironment immune status.

### TECHNICAL FIELD OF THE INVENTION

This invention is related to the area of therapeutic antibodies that are effective in the specific targeting of mesothelin-expressing cancers in immunoproficient and immunosuppressed tumor microenvironments. In particular, it relates to methods, kits, and compositions containing antibody-based agents with improved therapeutic efficacy in inhibiting cancer growth irrespective of microenvironment immune status.

### BACKGROUND OF THE INVENTION

The mechanism of tumorigenesis involves the combined accumulation of mutated genes that enhance dysregulated cell growth and the generation of immune avoidance mechanisms that enable their survival within an affected patient. Cellular (primarily referring to T-cell mediated) and humoral (primarily referring to antibody-mediated) immunity are the major mechanisms by which vertebrate host organisms defend against infectious pathogens and dysregulated host cells. Over the past few years, the use of immune checkpoint inhibitors that can overcome suppressed cellular-mediated immunity have demonstrated robust effects in unleashing activated CD8⁺ T-cell killing against subsets of tumors (Hodi F S, et al. N Engl J Med 363:711-723, 2010). Recent translational findings have shown tumors also produce factors that suppress humoral-immune pathways that in turn suppress the tumor-killing effects by antibody-mediated mechanisms, such as antibody dependent cellular cytotoxicity (ADCC), complement mediated cytotoxicity (CDC) and opsonization (Vergote I, et al. J Clin Oncol 34:2271-2278; Kline J B, et al. J Clin Oncol 5:15, 2018; Wang W et al. Cytogenet Genome Res 152:169-179, 2017; Kline J B et al. Eur J Immunol. 48:1872-1882, 2018; Dai S, et al. PLos Pathog 9:e1003114, 2013; Melero I, et al. Nat Reviews Cancer 7:95-106, 2007). The factors that suppress humoral-immune pathways inhibit the effects of clinically used therapeutic antibodies that have been reported to exhibit their tumor-killing effects through ADCC and CDC, such as, but not limited to, rituximab, obinutuzumab, trastuzumab, pertuzumab, cetuximab, alemtuzumab, as well as a number of experimental antibodies (DiLillo D J, Ravetech J V, Cancer Immunol Res 3:704-713, 2015; Ruck T, et al. Int J Mol Sci 16:16414-16439, 2015; Pelaia C, et al. Biomed Res Int 4839230:1-9, 2018; Zhou X, et al. Oncologist 13:954-966, 2008; Hsu Y F, et al. Mol Cancer 9:-8, 2010; Spiridon C I, et al. Clin Cancer Res 8:1720-1730, 2002; Kline J B, et al. Eur J Immunol 48:1872-1882, 2018; Yamashita-Kashima Y, et al. Clin Cancer Res 17:5060-5070, 2011). Antibody-mediated humoral immune responses are governed by the coordination of engagement of antibodies and cell surface antigens. When this interaction is optimal, cell surface bound antibodies engage with Fc-γ-activating receptors on Natural Killer (NK) or dendritic/myeloid/monocytic cells (any cell that participates in ADCC is referred to in this document as an “immune-effector cell”). This engagement initiates ADCC as well as engages with the C1q complement initiating protein to cause death of antibody-bound cells via the classical complement CDC pathway as well as through opsonization via phagocytes (Reuschenbach M, et al. Cancer Immunol Immunother 58:1535-1544, 2009). Inhibitors of the humoral immune response reduce the ability of therapeutic antibodies to use these mechanisms and in turn decreases their therapeutic efficacy (Wang W, et al. Cytogenet Genome Res 152:169-179, 2017; Kline J B, et al. Eur J Immunol 48:1872-1882, 2018; Felder M, et al. Gyn Oncol 152:618-628, 2019).

MUC16/CA125 protein (referred to here as CA125) suppresses humoral immune responses by binding to negative immune regulatory receptors of the SIGLEC family to suppress NK cell activation (Belisle J A, et al. Mol Cancer 9:1476-4598, 2010), and by direct binding to a subset of IgG1, IgG3 and IgM type antibodies. The binding to the antibodies perturb the Fc region making it less effective for IgG1 and IgG3 type antibodies to engage with Fc-γ-activating receptors FCGR2A (also referred to as CD32a) and FCGR3A (also referred to as CD16a) on immune-effector cells and/or perturbs the ability of all three antibody classes to engage with complement-mediating proteins, including C1q (Pantankar M S, et. al. Gyncol Oncol 99:704-713, 2005; Kline J B, et al. OncoTarget 8:52045-52060, 2017; Kline J B, et al. J. Clin. Oncol. 5:15, 2018; Wang W, et al. Cytogenet Genome Res 152:169-179, 2017; Kline J B, et al. Eur J Immunol. 48:1872-1882, 2018). These studies include clinical trials of experimental anti-cancer antibodies that rely on immune-effector mechanisms for their pharmacologic activity. In these clinical studies, CA125 levels have been found to correlate with clinical outcomes (Vergote I, et al. J Clin Oncol 34:2271-2278, 2016; Nicolaides N C, et al. Cancer Biol Ther 13:1-22, 2018). In studies involving the clinically used rituximab antibody in patients with follicular lymphoma, 31.4% improvement was observed in 5-year progression-free survival when CA125 levels were in the normal range compared to those with CA125 levels above the normal range (Prochazka V, et al. Int J Hematol 96:58-64, 2012). There is a continuing need in the art to develop agents that can effectively kill tumors with immunoproficient as well as immunosuppressed microenvironments.

Mesothelin is a cell surface protein that is over-expressed by a number of tumor types including mesothelioma, lung, pancreatic, ovarian, colorectal, cholangio, gastric and endometrial carcinomas. Several of these tumor types have been found to have humoral immune suppression, which potentially diminishes the efficacy of antibody-based, anti-mesothelin therapies (Rump A, et al. J Biol Chem 279:9190-9198, 2004; Hassan R, et al. Cancer Immunol 7:20-30, 2007; Kaneko 0, et al. J Biol Chem 284:3739-3749, 2009; Hassan R. Lung Cancer 68:455-459, 2010; Kelly R J, et al. J Clin Oncol suppl 32:61, 2014). In particular lung, ovarian, pancreatic, and mesothelioma cancers have been reported to express the humoral immunosuppressive CA125 protein (Vergote I, et al. J Clin Oncol 34:2271-2278, 2016; Nicolaides N C, et al. Cancer Biol Ther 13:1-22, 2018; Liu L, Oncotarget. 7:5943-5956, 2016; Cedres S, et. al. Clin Lung Cancer 12:172-179, 2011; Emoto S, et al. Gastric Cancer 15:154-161, 2012). Nicolaides et al (2018) reported humoral immunosuppression on findings from a Phase 2 clinical trial in first-line mesothelioma using the anti-mesothelin antibody amatuximab plus standard-of-care. In that trial patients treated with amatuximab with elevated CA125 had worse progression-free survival (PFS) and overall survival (OS) outcomes than those with low levels of CA125, thereby supporting the notion that anti-mesothelin antibodies relying on humoral immune function will provide lower clinical benefit in humoral immunosuppressed cancers such as those listed above. Alternative strategies to develop new antibody-based agents are required to potentially overcome this mechanism and offer patients with and without humoral suppressed cancers wider therapeutic options. There is a need in the art for compositions and methods that are effective in killing mesothelin-expressing tumor types exhibiting humoral immunosuppressed phenotypes (e.g., express the immunosuppressive CA125 protein, etc.) as well as those that are immunoproficient.

### SUMMARY OF THE INVENTION

One embodiment is an antibody-drug conjugate (ADC). It comprises an anti-mesothelin antibody comprising complementary determining regions (CDRs) with amino acid sequence shown in SEQ ID NOs: 7-12. One such anti-mesothelin antibody comprises SEQ ID NO:1 and SEQ ID NO: 2. The ADC also comprises a topoisomerase inhibitor.

Another embodiment is a method for employing as part of an antibody-drug conjugate (ADC) an anti-mesothelin antibody that does not bind CA125. The anti-mesothelin antibody comprises CDRs with amino acids shown in SEQ ID NOs: 7-12. The cellular uptake of the anti-mesothelin antibody is greater than the cellular uptake of anti-mesothelin antibodies that bind CA125, either in the soluble or membrane-bound form. One such anti-mesothelin antibody that does not bind CA125 which can be used comprises the amino acid sequences of SEQ ID NO: 1 and SEQ ID NO: 2. The method comprises administering the ADC to a human in need of anti-mesothelin therapy. The method may also comprise using the ADC to detect a mesothelin epitope in target cells.

Another embodiment is a method to treat a cancer patient who has a mesothelin-expressing tumor. The patient is treated by administering to the patient an antibody-drug conjugate (ADC) comprising an anti-mesothelin antibody comprising CDRs with amino acids shown in SEQ ID NOs: 7-12 and a topoisomerase inhibitor. One such anti-mesothelin antibody which can be used comprises SEQ ID NO:1 and SEQ ID NO: 2.

Another embodiment is a bispecific antibody (BSP) comprising a mesothelin-binding portion comprising amino acid sequences SEQ ID NOs: 7-12, and a human cell surface antigen CD3-binding portion.

Another embodiment is a method to treat a mesothelin-expressing cancer in a patient. A bispecific antibody (BSP) comprising a mesothelin-binding portion comprising amino acid sequences SEQ ID NOs: 7-12 and a human cell surface antigen CD3-binding portion is administered to the patient, thereby treating the mesothelin-expressing cancer.

One aspect of the invention is an antibody comprising the amino acid sequences shown in SEQ ID NO: 1 [MES light chain] and SEQ ID NO: 2 [MES heavy chain]. Because significantly less of the antibody is bound by immunosuppressive CA125 protein, the antibody enjoys enhanced internalization. This is particularly helpful for ADC-mediated tumor cell killing when the antibody is part of an antibody-drug conjugate.

Another aspect of the invention is an antibody comprising the amino acid sequences shown in SEQ ID NO: 1 [MES light chain] and SEQ ID NO: 2 [MES heavy chain] where the antibody is conjugated to a cytotoxic compound that is selected for its ability to kill immunocompetent as well as immunosuppressed mesothelin-expressing cancer cells. The cytotoxic compound may be a topoisomerase inhibitor, microtubule inhibitor, alkylating agent or kinase inhibitor. The conjugate is referred to as “MES-ADC.”

Another aspect of the invention is a stable cell line containing a mammalian expression vector with nucleotide sequences shown in SEQ ID NO: 19 [MES light chain] and SEQ ID NO: 5 [MES heavy chain] encoding the parental MES-1 antibody that is produced by the cell line and optionally thereafter chemically linked to a cytotoxic agent.

Another aspect of the invention is a method to treat a patient with mesothelin-expressing cancer cells that also expresses an elevated level of CA125 compared to a population of healthy humans. A MES-ADC antibody is administered to the patient. The MES-ADC antibody is comprised of the amino acid sequences SEQ ID NO:1 and SEQ ID NO: 2 and is linked to the cytotoxic topoisomerase inhibitor SN38. The antibody-cytotoxin is optionally covalently linked by a cleavable linker.

Yet another aspect of the invention is a method to treat a patient with mesothelin-expressing cancer cells who also expresses an elevated level of CA125 compared to a population of healthy humans. A MES-ADC antibody is administered to the patient. The MES-ADC antibody is comprised of the amino acid sequences SEQ ID NO: 1 and SEQ ID NO: 2 and is linked to the cytotoxic topoisomerase inhibitor PNU159682. The antibody-cytotoxin may be covalently linked by a cleavable linker.

In one aspect, a MES-ADC comprises the antibody comprised of SEQ ID NO: 1 and SEQ ID NO: 2 linked to the cytotoxic PNU159682 or SN38 topoisomerase inhibitors. Two or more cytotoxins may be bound to the antibody via chemical ligation through the linking to free cysteines on said antibody that are generated by partial reduction. The cysteines may be native to the immunoglobulin sequence or engineered into the parent antibody sequence.

In another aspect of the invention, the MES-ADC chemical ligation utilizes a cleavable linker, such as but not limited to Val-Cit-PAB, MA-PEG4-VC-PAB-DMAE, Fmoc-Val-Cit-PAB, Fmoc-Val-Cit-PAB-PNP, MC-Val-Cit-PAB-PNP, Phe-Lys(Trt)-PAB, Fmoc-Phe-Lys(Trt)-PAB, Fmoc-Phe-Lys(Trt)-PAB-PNP, Ala-Ala-Asn-PAB TFA salt, Fmoc-Ala-Ala-Asn-PAB-PNP, Fmoc-Gly3-Val-Cit-PAB, Fmoc-Gly3-Val-Cit-PAB-PNP, Py-ds-Prp-OSu, Py-ds-dmBut-OSu, Py-ds-dmBut-OPFP, Py-ds-Prp-OPFP, PEG8-triazole-PABC-peptide-mc, etc., whose chemical structures are known in the art.

In another aspect of the invention, the MES-ADC chemical ligation utilizes a non-enzymatic cleavable linker, such as but not limited to SMCC, MAL-HA-OSu, MAL-di-EG-OPFP, MAL-tri-EG-OPFP, MAL-tetra-EG-OPFP, N3-di-EG-OPFP, N3-tri-EG-OPFP, N3-tetra-EG-OPFP, ALD-BZ-OSu, ALD-di-EG-OSu, ALD-tetra-EG-OSu, ALD-di-EG-OPFP, ALD-tetra-EG-OPFP, MC-EDA, PHA-di-EG-OPFP, PHA-tetra-EG-OPFP, etc., whose chemical structures are known in the art.

In another aspect of the invention, the linker is optimized for (a) antibody conjugation to cytotoxin; (b) stability in systemic circulation, organ parenchyma/stroma and tumor microenvironment; and/or (c) improved killing of immunosuppressed tumors by MES-ADC.

In another aspect of the invention, the cytotoxin is linked to one or more lysine residues contained with the light and heavy chains of an anti-mesothelin antibody. One such antibody comprises the amino acid sequences shown in SEQ ID NO: 1 and/or SEQ ID NO: 2.

In another aspect of the invention, the cytotoxin is linked to the C-terminus of the heavy chain contained in SEQ ID: 2 via trans-amidation.

Another aspect of the invention is a bispecific antibody comprising the amino acid sequences shown in SEQ ID NO: 3 and SEQ ID NO: 2, i.e., single chain anti-CD3 fused to MES-1 light chain and MES-1 heavy chain, respectively. The bispecific antibody is able to kill immunocompetent as well as immunosuppressed mesothelin-expressing cancer cells. The single chain anti-CD3 antibody recognizes a cell surface antigen expressed on CD3⁺ and/or CD8⁺ lymphocytes. The bispecific antibody is referred to herein as MES-BSP, i.e., a bispecific antibody that targets mesothelin.

Another aspect of the invention is a bispecific antibody that targets mesothelin, in which the antibody is not bound by the CA125 immunosuppressive protein. One such antibody comprises the amino acid sequences of SEQ ID NO: 3 and SEQ ID NO: 2. The bispecific antibody can be used to treat a mesothelin-expressing cancer or other disease related to mesothelin expression.

Another aspect of the invention is a stable cell line containing one or more mammalian expression vectors with nucleotide sequences encoding the antibody shown in SEQ ID NO: 19 [MES light chain cDNA] and SEQ ID NO: 5 [MES heavy chain cDNA] genetically linked to a second antibody.

Another aspect of the invention is a method to treat a patient with mesothelin-expressing cancer cells that also expresses an elevated level of CA125 compared to a population of healthy humans. A MES-BSP antibody is administered to the patient. The MES-BSP antibody is comprised of the amino acid sequences SEQ ID NO: 3 and SEQ ID NO: 2; the bispecific antibodies recognize mesothelin as well as the human CD3 protein.

Another aspect of the invention is a method to treat a patient with mesothelin-expressing cancer cells that also expresses an elevated level of CA125 compared to a population of healthy humans. A MES-BSP antibody is administered to the patient. The MES-BSP antibody comprises amino acid sequences SEQ ID NO:1 and SEQ ID NO: 2 and an antibody that can recognize the human CD8 protein.

In yet another aspect of the invention, an antibody comprising the amino acid sequence of SEQ ID NO:1 and the amino acid sequence of SEQ ID NO: 2 is fused to a single chain antibody comprising the amino acid sequence of SEQ ID NO: 6, which is fused to the N-terminus of the human IgG1 light chain (SEQ ID NO: 1) and/or the N terminus of the IgG heavy chain (SEQ ID: 2), leading to a bispecific antibody capable of binding mesothelin (Entrez Gene ID: 10232) and CD3 epsilon (CD3E) protein (Entrez Gene ID: 916), referred to here as MES-BSP.

In another aspect of the invention, the fusion of the amino acid sequence of SEQ ID NO: 1 and the amino acid sequence of SEQ ID NO: 6 that are tethered via a linker results in a light chain fusion polypeptide consisting of SEQ ID NO: 3 which is combined with SEQ ID NO: 2 to generate a functional MES-BSP that can bind (a) mesothelin on target cancer cells in the presence or absence of CA125 and (b) CD3E on lymphoid cells, leading to killing of target cells. The linker may be an amino acid, a polypeptide or a non-biological chemical compound.

Yet another aspect of the invention is a MES-BSP that comprises complementarity determining regions (CDRs) having the amino acid sequence of SEQ ID NOs: 7, 8, 9; SEQ ID NOs: 10, 11, 12; SEQ ID NOs: 13, 14, 15; and SEQ ID NOs: 16, 17, 18 in which up to 3 amino acids may be altered within one or more CDRs.

In another aspect of the invention, the linker in SEQ ID NO: 3 is optimized for (a) canonical antibody formation with the IgG1 heavy chain (SEQ ID NO: 2) and/or (b) improved killing of immunosuppressed tumors by MES-BSP, whereby the linker comprises any combination of the 20 naturally occurring amino acid units to maximize spatial distancing of the anti-mesothelin light chain (SEQ ID NO: 1) and anti-CD3E single chain (SEQ ID NO: 6).

In another aspect of the invention, MES-BSP comprises the anti-CD3E single chain genetically linked to the N-terminus of the heavy chain of SEQ ID: 2. The linker is optimized for (a) canonical antibody formation with the IgG1 light chain (SEQ ID NO: 1) and/or (b) improved killing of immunosuppressed tumors by MES-BSP, whereby the linker comprises any combination of the 20 naturally occurring amino acid units to maximize spatial distancing of the anti-mesothelin heavy chain (SEQ ID NO: 2) and anti-CD3E single chain (SEQ ID NO: 6).

Another aspect of the invention is a stable cell line containing mammalian expression vectors with nucleotide sequences shown in SEQ ID NO:5 and SEQ ID NO: 4 encoding the MES-1 antibody genetically linked to a second antibody.

Another aspect of the invention is an antibody that has the CDR amino acid sequences of SEQ ID NOs: 7-12 grafted onto a rabbit IgG backbone (referred here as rMES-1). The antibody is not bound by CA125 protein.

Another aspect of the invention is a method for monitoring a tumor expressing mesothelin in a patient employing an antibody that is not bound by CA125. A body fluid or tissue sample from the patient is contacted with an antibody containing CDRs SEQ ID NOs: 7-12, such as antibody rMES-1. Patients that are positive for mesothelin may be treated with MES-ADC or MES-BSP.

Another aspect of the invention, a kit is provided for treating immunoproficient and immunosuppressive mesothelin cancers. The kit comprises an antibody containing the CDRs SEQ ID NOs: 7-12 preferably grafted onto a rodent IgG backbone. The antibody may be used to monitor tumor for mesothelin expression via immunohistochemistry (IHC) on biopsied tissue or via flow cytometry on circulating tumor cells (CTCs). Upon detection of a positive signal, a patient may be treated with MES-ADC comprising amino acid sequences of SEQ ID NO: 1 and SEQ ID NO: 2 chemically linked to a cytotoxin, and the cytotoxin may be a topoisomerase inhibitor. Both the diagnostic and therapeutic antibodies may be provided in the kit.

In another aspect of the invention, a kit is provided for treating immunoproficient and immunosuppressive mesothelin cancers. The kit comprises an antibody containing CDRs according to SEQ ID NOs: 7-12 preferentially grafted onto a rodent IgG backbone. The antibody can be used to monitor tumor for mesothelin expression via IHC on biopsied tissue or via flow cytometry on circulating tumor cells (CTCs). Upon detection of a positive signal, the patient may be treated with an MES-BSP consisting of SEQ ID NO: 2 and SEQ ID NO: 3. Both the diagnostic and therapeutic antibodies are provided in the kit.

Another aspect of the invention is the use of a MES-ADC in which the MES-ADC is administered as a single agent or in combination with standard-of-care chemotherapies to patients in which a subset express CA125. CA125 expression may be determined using serum analysis or biopsy via methods used by those known in the art.

Another aspect of the invention is the use of a MES-BSP in which the MES-BSP as a single agent or in combination with standard-of-care chemotherapies is administered to patients in which a subset express CA125. CA125 expression is determined using serum analysis or biopsy via methods known in the art.

These and other aspects of the invention which will be apparent to those skilled in the art upon reading the specification provide methods, compositions, and kits for use in improving therapeutic responses in patients with mesothelin-expressing cancers irrespective of the immune status of the tumor microenvironment. The refractory nature of the MES-ADC and/or MES-BSP agents with regard to CA125 inhibition contribute to this improvement in treatment.

### BRIEF DESCRIPTION OF THE DRAWINGS

FIG. 1A. Screening for anti-mesothelin antibodies not bound by the immunosuppressive CA125 protein; identification of MES-1 antibody (SEQ ID NO: 1 and 2). Briefly, 96-well ELISA plates were coated with 15 KU/mL of soluble CA125, 1 μg/mL of recombinant human mesothelin protein as a positive control and 1 μg/mL of human serum albumin (HSA) as a negative control and probed with 2.5 μg/mL of different anti-mesothelin antibodies to determine if they are bound by CA125. Wells were washed and detected for binding via a secondary anti-human or anti-rodent Fc-horseradish peroxidase (HRP) conjugated antibody and 3,3′,5,5′-Tetramethylbenzidine (TMB) substrate. Reactions were stopped with 0.1N H₂SO₄ and wells quantified using a multi-well plate reader (Varioskan™, ThermoFisher) at 450 nm. As shown, the antibody MES-1 consisting of SEQ ID NOs: 1 and 2 [Ab-3 (lane 3)] was not bound by CA125 while the other four anti-mesothelin antibodies tested were all bound by CA125. Values were determined from the average of triplicate wells. Statistical analysis was done using student's T-test. FIG. 1B. These results demonstrate the enhanced uptake of anti-mesothelin antibodies that are not bound by CA125 (refer to FIG. 1A Ab-3) in contrast to those that are bound by CA125 (refer to FIG. 1A Ab-4). To measure antibody internalization, the pHrodo™ fluorescent assay (ThermoFisher) was employed, whereby pH sensitive fluorescent dyes were conjugated to the CA125 refractory Ab-3 (also referred to MES-1 here in) and the CA125 sensitive Ab-4 antibodies. Antibodies were tested for uptake by incubating each with the human ovarian cancer cell line OVCAR3 that express mesothelin and membrane bound CA125. The parental line was used to generate an isogenic CA125 knockdown line via shRNA (referred to as OVCAR3-KO). Both Ab-3 and Ab-4 uptake were measured in replica in black 96-well microplates over the course of 24 hours measuring intracellular fluorescence on a Varioskan™ plate reader. As shown, Ab-3 (MES-1) had similar uptake in both cell lines while Ab-4 had similar uptake to Ab-3 in OVCAR-KO cells but not the parental OVCAR3 which expresses CA125 demonstrating an negative impact of Ab-4 uptake via CA125 in contrast to the CA125 refractory Ab-3. Statistical analysis was done using a two-sided student's T-test.

FIGS. 2A-C. Cytotoxic payloads and linkers examined for developing reformatted MES-1 antibody for optimal killing of immunoproficient and immunosuppressed mesothelin expressing cancer cells. FIG. 2A provides chemical structures of various types of cytotoxic payloads (i.e., microtubule inhibitors, DNA alkylating agents, topoisomerase inhibitors, and protein kinase inhibitors, see, e.g., Yaghoubi S, et al. J Cell Physiol 235:31-64, 2019; Wang R E, et al. J Am Chem Soc 137:3229-3232, 2015) tested against immunosuppressive and immunoproficient mesothelin expressing target cells. FIG. 2B provides chemical structures of various enzymatic and non-enzymatic cleavable and non-cleavable linkers tested to generate MES-ADCs against immunosuppressive and immune-proficient mesothelin expressing target cells. FIG. 2C provides an overview of the cytotoxicity of potential ADC payloads against mesothelin expressing cancer cells or control cell lines (Table 1). Briefly, 96-well tissue culture plates were seeded with 5,000 cells/well of mesothelin-positive NCI-N87 (CA125⁺ gastric cancer), SW1990 (CA125⁺ pancreatic cancer), HAY (CA125⁺ mesothelioma), YOU (CA125⁻ mesothelioma), OVCAR3 (CA125⁺ ovarian cancer) and A549 (mesothelin negative lung cancer) cells in 100 μLs of RPMI plus 7.5% heat inactivated fetal bovine serum (FBS), and 0.0001 to 500 ng/mL of cytotoxin or negative control. Cultures were incubated for 72 hours at 37° C. in 5% CO₂ then quantified for viability using crystal violet staining. Dried stained wells were solubilized using 1% SDS in phosphate buffer saline (PBS) and quantified by colorimetric densitometry on a Varioskan™ multi-well plate reader at 570 nm. Several cytotoxins tested were able to significantly kill mesothelin target cells irrespective of CA125 expression as well as non-mesothelin expressing control cells A549 and CHO (not shown). As shown, the microtubule inhibitor MMAE, as well as the topoisomerase inhibitors SN38 and PNU159682 were found to have the best potency with EC50s ranging from 0.008 to 5 ng/mL. Experiments represent a minimum of triplicate wells. Statistical analysis was done using a two-sided Student' T-test.

FIGS. 3A-3C. MES-1 and MES-ADC composition, purification and structural analysis. FIG. 3A, analysis of CHO-GS produced and protein A purified MES-1 antibody. Size exclusion (SEC) HPLC analysis of purified MES-1 demonstrates highly homogeneous antibody species, which is a prerequisite for producing homogeneous antibody drug conjugates with a desired drug:antibody ratio (DAR). FIG. 3B provides a schematic overview of SN38-MES-ADC and PNU-MES-ADC compositions using cleavable linkers. FIG. 3C, SN38-MES-ADC and PNU-MES-ADC DAR homogeneity as determined by hydrophobic interaction chromatography (HIC-HPLC) and size exclusion chromatography (SEC-HPLC). Both MES-ADCs were generated through partial reduction of MES-1 and cysteine ligation. DAR was calculated from the HIC peak area and retention time. As shown, the SN38-MES-ADC and PNU-MES-ADC provide representative profiles of the reproducibility for generating both of these ADCs with a desired DAR of 2-6.

FIGS. 4A-4E. MES-ADC conjugate formats and target cell killing in vitro and in vivo. FIG. 4A, target cell killing via MES-ADCs containing the two most potent cytotoxins, SN38 and PNU159682 observed from the free-cytotoxin toxicity assays in FIG. 2C. MES-ADCs were generated using cleavable linkers and tested for killing of the various target and control cell lines listed in Table 1. Assays were conducted as described in FIG. 2C. Briefly, 96-well tissue culture plates were seeded with 5,000 cells/well of mesothelin-positive NCI-N87, SW1990, HAY, YOU, OVCAR3, CHO-MES as well as mesothelin negative A549 and CHO cells as negative controls. Cells were plated in 100 μLs of RPMI plus 7.5% heat inactivated fetal bovine serum (FBS), with varying MES-ADC concentrations via limiting dilution (ranging from 0.01 to 100 ng/mL) or negative control compounds. Cultures were incubated for 72 hours at 37° C. in 5% CO₂ then quantified for viable cells via crystal violet staining. Dried stained wells were solubilized using 1% SDS in PBS and quantified by colorimetric densitometry on a Varioskan™ multi-well plate reader at 570 nm. As shown, the SN38-MES-ADC and PNU-MES-ADC had the most significant cell killing across all target cells expressing mesothelin irrespective of CA125 expression status, while the mesothelin-negative lines A549 or CHO (not shown) were unaffected, demonstrating selectivity and potency of both ADCs against mesothelin-expressing cells. We next tested the lead MES-ADCs in different linker formats (FIG. 4B). The NCI-N87 and SW1990 cells were used as target cells and PNU-MES-ADC was used as a representative MES-ADC for potency analysis in an enzymatic cleavable or non-enzymatic cleavable linker format. Cells were plated and grown as described above with varying concentrations of each PNU-MES-ADC via limiting dilution. As shown, the PNU-MES-ADC in an enzymatic cleavable format was significantly more potent (grey and blue lines) than the PNU-MES-ADC in non-enzymatic cleavable format. A549 mesothelin negative cells were unaffected by either ADC (not shown), again demonstrating the MES-ADC potency and target specificity. FIG. 4C: the NCI-N87, SW1990 tumor cell lines as well as a panel of PDX-derived tumors were screened for mesothelin expression via IHC using the rMES-1 detector antibody and a CA125 commercial antibody to ensure the expression profiles of these two key proteins are maintained in vivo. Both the NCI-N87 and SW1990 cell lines removed as tumor fragments from xenografts maintained expression of both proteins. After screening of multiple samples, two PDX tumor fragments were identified as having similar levels of mesothelin expression, whereby one also expressed robust levels of CA125 (mesothelioma #PXF1118) while the other had undetectable expression (non-small cell lung adenocarcinoma (NSCLC) #LXFA983). FIG. 4D: testing of SN38-MES-ADC and PNU-MES-ADCs in vivo. First, 1×10⁷ tumor cells were injected into the flank of multiple athymic nude mice for SW1990 cells or SCID mice for N87 cells. Upon establishment of measurable tumor lesions (>100 mm³), mice were grouped and tested for tumor killing using the cleavable and non-cleavable PNU-MES-ADC formats, SN38-MES-ADC and PBS as negative control. As shown, both the cleavable SN38-MES-ADC and PNU-MES-ADC formats were found to be significantly more efficacious in vivo (Top panel: SN38-MES-ADC P<0.049 at 10 mg/kg and P<0.0003 at 20 mg/kg; Bottom panel: PNU-MES-ADC P<0.011) than the non-enzymatic cleavable format (not shown), reflecting the in vitro target cell killing effect. FIG. 4E: the cleavable PNU-MES-ADC was tested against the immunoproficient LXFA983 (CA125 low) and the immunosuppressed PXF1118 (CA125 high) PDX-derived tumor xenografts to determine efficacy against these distinct tumor types. Tumor fragments were implanted into the flank of athymic nude mice and treated with PNU-MES-ADC or PBS control. As shown, PNU-MES-ADC was equally effective in killing and causing regression of both tumors irrespective of microenvironment immune (CA125 expression) status (P<0.012. Experiments represent a minimum of triplicate wells for in vitro assays and a minimum of five subjects for in vivo assays. Statistical analysis was done using a two-sided Student' T-test.

FIG. 5. Single and multiple dosing and anti-tumor effects of cleavable PNU-MES-ADC on CA125 positive, mesothelin-expressing PXF1118 tumors. Tumor fragments were implanted into the flank of athymic nude mice and grown to an average size of 100 mm³. Mice with similar sets of tumor sizes were grouped into 4 sets of 6 mice each and treated with PBS, 0.25 mg/kg PNU-MES-ADC or 30 μg/kg of free PNU159682 treated on day 1, 8 and 15 after randomization. A fourth arm using 0.75 mg/kg PNU-MES-ADC was given a single dose at day 1 after randomization and mice were monitored for tumor growth over more than 50 days. As shown, single dose PNU-MES-ADC was equally effective in killing and causing regression of tumors and maintaining a durable response greater than 50 days similar to the multi-dosed PNU-MES-ADC treated mice in contrast to mice treated with PBS or free drug (P<0.0061). Experiments represent six subjects per group. Statistical analysis was done using a two-sided Student' T-test.

FIGS. 6A-6B. ADCC activity of MES-BSP and control antibodies against immunosuppressed cancer cells. To test the effects of MES-BSP immune-mediated targeting of immunosuppressed cancer cells, ADCC assays using primary peripheral blood mononuclear cells (PBMCs) and Jurkat-CD16a ADCC reporter assays were employed. FIG. 6A, MES-BSP, MES-1 and anti-mesothelin meso-Ab-4 (refer to FIG. 1A, lane 4) antibodies were tested for PBMC immune-mediated killing of the human OVCAR3 ovarian cancer cell line, which expresses mesothelin and produces high amounts of the immunosuppressive CA125 protein. Briefly, 10,000 OVCAR3 cells were plated per well in 96-well black plates overnight in 65 μLs RPMI plus 7.5% fetal bovine serum and 1% L-glutamine (R7.5). The next day, 35 μL of various concentrations of MES-BSP, MES-1 and meso-Ab-4 plus 2.5×10⁷ PBMCs in R7.5 media were added to each well and plates were incubated at 37° C. in 5% CO₂ for 72 hours. Wells were then washed three times with 250 μLs R7.5 media to remove PBMCs and the adherent OVCAR3 target cell viability was quantified via Cell Titer GLO® as per manufacturer's directions (Promega) on a Varioskan™ luminescent plate reader. As shown, while both MES-1 and MES-BSP antibodies showed tumor cell killing in contrast to meso-Ab-4, the MES-BSP had significantly the highest target cell killing, thus demonstrating the ability of MES-1 in BSP format to provide enhanced killing over parental MES-1 alone when utilizing immune-mediated targeting. To determine the impact of the immunosuppressive CA125 protein on ADCC activity, the MES-1 and meso-Ab-4 antibodies were tested against OVCAR3 cells and an OVCAR3-CA125 knockdown (OVCAR-KO) cell line generated using the shRNA vector TRCN0000262686 (Sigma-Aldrich) as previously described (Kline J B, et. al. OncoTarget 8:52045-52060, 2017) and the Jurkat-CD16a ADCC reporter cell line following the manufacturer's instructions (Promega Corp). The Jurkat ADCC system employs a luciferase readout, whereby luciferase signal intensity represents ADCC activity of an antibody against a target cell. As shown in FIG. 6B, while MES-1 had significantly higher ADCC activity against OVCAR3 than meso-Ab-4, both the MES-1 and meso-Ab-4 had similar ADCC activity against the OVCAR-KO cell line, thus demonstrating the utility of the naturally CA125 refractory MES-1 parental antibody in alternative formats MES-1 (i.e. ADC or BSP) as an agent capable of effectively killing immunosuppressive tumor cells. MES-BSP was equally effective against both OVCAR3 and OVCAR-KO lines. All experiments were performed in triplicate. Statistical analysis were performed using a two-sided Student's T-test.

FIG. 7. MES-BSP was tested in vivo using humanized PBMC nude mice and a mesothelin positive, CA125 expressing human mesothelioma cell line. Athymic nude mice were implanted with 8×10⁶ mesothelioma cells on day 0. On day 11 when average lesions where ˜50 mm³, 1×10⁷ human peripheral blood mononuclear cells (PBMCs) were administered intraperitoneally. The following day, 3 mg/kg of MES-BSP or PBS was administered 3×QOD for 3 weeks and tumors were monitored for growth (N=5). As shown, MES-BSP caused 70% reduction of CA125-positive mesothelioma tumors as compared to control treated mice (P=0.033). Statistical analysis was done using a two-sided Student's T-test.

### DETAILED DESCRIPTION OF THE INVENTION

The inventors have developed new therapeutic agents capable of effectively killing mesothelin-positive cancer cells irrespective of the tumor cell microenvironment immune status. While not wanting to be limited to any particular theory or mechanism of action, applicants believe that amino acid sequences encoded by SEQ ID NOs: 1 and 2 (referred here in as MES-1 antibody) as well as SEQ ID NOs: 2 and 3 (referred here as MES-BSP antibody) are refractory to binding by the immunosuppressive CA125 protein which negatively affects both (a) antibody mediated immune responses (Kline J B, et. al. OncoTarget 8:52045-52060, 2017; Kline J B, et al. Eur J Immunol 48:1872-1882, 2018; Kline J B, et al. J Clin Oncol 5:15, 2018; Nicolaides N C, et al. Cancer Biol Ther 13:1-22, 2018) as well as (b) antibody drug conjugate (ADC) uptake and delivery of cytotoxic agents internally, which is a prerequisite for robust target cell killing via ADCs (Nicolaides N C, et. al. U.S. patent application Ser. No. 16/984,444; Chalouni C and Doll S. J Exp Clin Cancer Res 37:20-32, 2018). This immunosuppressive mechanism appears to involve the direct binding of CA125 to affected antibodies (Nicolaides N C, et al. Cancer Biol Ther 13:1-22, 2018).

In addition, the application provides methods to identify additional antibody-based therapies that are effective against tumors irrespective of their microenvironment immune status. These include the testing of parental antibodies for binding to tumor produced immunosuppressive proteins such as CA125. Such antibodies can be formatted in antibody-drug conjugates or bispecific formats and empirically tested to optimize effectiveness at killing tumors with immunosuppressed microenvironments by using various payloads as well as genetic fusions and chemical linkers.

The methods and kits described here may be used to monitor and confirm the eligibility of patients expressing mesothelin who are suitable for treatment with MES-ADC or MES-BSP by testing patient tumor cells with an antibody containing CDR amino acid sequences SEQ ID NOs: 7-12 using methods for antigen expression testing known in the art.

The methods, compositions, and kits may be divided into two categories of antibody formatting. In one category, a MES-ADC is developed that comprises immunoglobulin light (SEQ ID NO: 1) and heavy (SEQ ID NO: 2) chains that are chemically linked to a cytotoxic agent. Direct binding by CA125 to the antibody component is low or null. The number of cytotoxin moieties per antibody molecule [referred to as drug:antibody ratio (DAR)] may vary depending on the methods of conjugation, with a minimum DAR of 2 and a maximum DAR of 12, with a preference of 2, 3, 4, 5, or 6. The cytotoxic agent may be, but is not limited to, the topoisomerase inhibitors SN38 or PNU159682. The cytotoxin moieties are linked to the antibody by a chemical or peptide linker. The linker may be of the cleavable or non-cleavable type, as known by those skilled in the art. The desired combination of linker, cytotoxin, and DAR can be empirically determined to optimize the activity of the MES-ADC on mesothelin tumor cell killing in vitro and/or in vivo. Tumor cell viability can be determined employing methods used in the art.

Suitable cytotoxins include without limitation those which are safe and preferably biodegradable. These include but are not limited to: Monomethyl Dolastatin 10, Auristatin E, Monomethyl Auristatin E (MMAE), Auristatin F, Monomethyl Auristatin F, HTI-286, Tubulysin M, Maytansinoid AP-3, Maytansinol, DM1, DM4, Boc-Val-Dil-Dap-OH, Boc-Val-Dil-Dap-Phe-Ome, Boc-Val-Dil-Dap-Doe, Boc-Val-Dil-Dap-Nrp, Boc-N-Me-Val-Val-Dil-Dap-OH, Tubulysin IM-1, Tubulysin IM-2, Tubulysin IM-3, Dasatinib, Duocarmycin SA, Duocarmycin TM, Duocarmycin MA, Duocarmycin DM, Nemorubicin, PNU-159682, Calicheamicin γ1, N-Acetyl-Calicheamicin γ1, α-Amanitin, PBD-dimer, etc. whose structures are known in the art.

Suitable linkers include without limitation those which are safe and preferably biodegradable. These include but are not limited to: Val-Cit-PAB, Fmoc-Val-Cit-PAB, Fmoc-Val-Cit-PAB-PNP, MC-Val-Cit-PAB-PNP, Phe-Lys(Trt)-PAB, Fmoc-Phe-Lys(Trt)-PAB, Fmoc-Phe-Lys(Trt)-PAB-PNP, Ala-Ala-Asn-PAB TFA salt, Fmoc-Ala-Ala-Asn-PAB-PNP, Fmoc-Gly3-Val-Cit-PAB, Fmoc-Gly3-Val-Cit-PAB-PNP, MAC glucuronide phenol, Py-ds-Prp-OSu, Py-ds-dmBut-OSu, Py-ds-dmBut-OPFP, Py-ds-Prp-OPFP, SMCC, MAL-HA-OSu, MAL-di-EG-OPFP, MAL-tri-EG-OPFP, MAL-tetra-EG-OPFP, MA-PEG4-VC-PAB-DMAE, MC-EDA, N3-di-EG-OPFP, N3-tri-EG-OPFP, N3-tetra-EG-OPFP, ALD-BZ-OSu, ALD-di-EG-OSu, ALD-tetra-EG-OSu, ALD-di-EG-OPFP, ALD-tetra-EG-OPFP, PHA-di-EG-OPFP, PHA-tetra-EG-OPFP, PEG8-triazole-PABC-peptide-mc, etc., whose chemical structures are known in the art.

One embodiment is an MES-ADC comprising the MES-1 antibody (SEQ ID NOs: 1 and 2) that has low CA125 binding and is covalently linked to the PNU159682 topoisomerase inhibitor by the MA-PEG4-VC-PAB-DMAE cleavable linker.

Another embodiment is an MES-ADC comprising the MES-1 antibody (SEQ ID NOs: 1 and 2) that has low CA125 binding and is covalently linked to the SN38 topoisomerase inhibitor by the MAC glucuronide phenol or PEG8-triazole-PABC-peptide-mc cleavable linkers.

In another category of antibody formatting, a MES-BSP (bispecific antibody) comprises immunoglobulin heavy chain (SEQ ID NO: 2) of MES-1 antibody and a chimeric light chain (SEQ ID NO: 3) that has low CA125 binding. The chimeric light chain is a genetic fusion (in amino to carboxyl order) of an anti-CD3 single chain antibody (SEQ ID NO: 6) fused to the MES-1 light chain (SEQ ID NO: 1). The anti-CD3 single chain antibody portion is genetically linked via a spacer comprising any one of the 20 known natural or modified amino acids, whereby the linker may be of two or more amino acids that separate the light chain from the single chain, as known in the art. A desired linker amino acid composition and length can be empirically determined to optimize the activity of the MES-BSP on mesothelin tumor cell killing in vitro and/or in vivo in the presence of human CD3⁺ lymphocytes. Tumor cell viability can be determined employing any of a variety of methods known in the art.

For therapeutic applications, the MES-ADC or MES-BSP can be administered as monotherapy or in combination with standard-of-care therapy.

Compositions can be formed in the course of conducting the methods. They may be pre-formed and packaged individually and provided to an entity that has a cytotoxin library to screen or one that can employ varying linkers to link the cytotoxin to the MES-1 antibody, for example. Similarly, the components of the assays and methods described here may be packaged together in a container and sold as a kit. The components of a kit need not be, but may be mixed together. They can be provided in separate containers or in a divided container, for example. Any selection of detector antibody, and the MES-ADC or MES-BSP described here may be formulated as a composition or kit.

While there are several known antibodies that are susceptible to CA125 immunosuppression, the compositions described here are useful for treating patients with mesothelin expressing cancers irrespective of tumor microenvironment immune status with MES-ADC or MES-BSP. Each of these has one or more of the MES-1 parental sequences (SEQ ID NOs: 1-3) that are refractory to CA125 binding and therefore effective in ADC internalization and killing when in MES-ADC format or effective in immune-related killing when in MES-BSP format.

In some instances, the MES-ADC may need to be formulated in liposomes to enhance its therapeutic window in patients with mesothelin-expressing cancers. Any liposome formulation for delivery of MES-ADC may be used to treat a patient. Conventional liposomes consist of a lipid bilayer composed of cationic, anionic, or neutral (phospho)lipids and cholesterol, which encloses an aqueous volume. Suitable compositions of liposomes include without limitation, the guanidinium-cholesterol cationic lipid bis (guanidinium)-tren-cholesterol (BGTC) combined with the colipid dioleoyl phosphatidylethanolamine (DOPE). Another example of a suitable liposome formulation is the aminoglycoside lipid dioleyl succinyl paromomycin (DOSP) associated with the imidazole-based helper lipid MM27. Liposomes can be sterically stabilized, using, for example, polyethylene glycol to coat a liposome.

We provide compositions, kits and methods for identifying patients with mesothelin-positive cancers and treating them with MES-ADC or MES-BSP. The methods may include a step of diagnosis of a patient's tumor for mesothelin expression using the CA125 refractory MES-1 detector antibody comprising CDRs SEQ ID NOs: 7-12. If positive in this assay, the tumor may be treated with MES-ADC (SEQ ID NOs: 1 and 2) linked to a cytotoxin that is optionally a topoisomerase inhibitor, or MES-BSP (SEQ ID NOs: 2 and 3). The diagnosis and therapeutic steps may be independent or performed in concert. In cancers where mesothelin is highly over expressed, the use of such prescreening may be optional.

Another embodiment is the MES-ADC containing CDRs (SEQ ID NOs: 7-12), whereby any of these CDR sequences may be modified by up to three amino acids individually or in combination as long as they remain CA125 refractory.

In another embodiment, the cytotoxin of the MES-ADC is SN38 or PNU159682 linked to a cleavable linker. The linker may be the PEG8-triazole-PABC-peptide-mc linker (C₅₀H₇₉N₉O₁₆) linked to SN38 (the whole construct referred to as SN38-MES-ADC-1), the MAC glucuronide phenol-linker linked to SN38 (the whole construct referred to as SN38-MES-ADC-2) or the MA-PEG4-VC-PAB-DMAE linker linked to PNU159682 (the whole construct referred to as PNU-MES-ADC).

In another embodiment the MES-1 light chain (SEQ ID NO: 1) is linked to the CD3 single chain (SEQ ID NO: 6) using an amino acid linker unit comprised of the amino acids GGGGS (SEQ ID NO: 20). The linker is comprised of one or more linker units. As an example but not wanting to be limited to length or amino acid composition, the MES-1 light chain and anti-CD3 single chain can be linked to one, two, three, or more units. Linker optimization can be determined by optimal killing of mesothelin target cell in the presence of CD3⁺ lymphocytes using any method used in the art to measure tumor cell killing as described here.

In yet another embodiment, a linker unit comprises any combination of the known natural or modified amino acids and any length that may genetically link the MES-1 light chain to the anti-CD3 single chain that can be empirically tested for optimizing tumor cell killing in mesothelin positive tumors with immunoproficient or -suppressed microenvironments and human CD3⁺ lymphocytes.

Another embodiment has the MES-BSP containing the amino acid sequences of SEQ ID NOs: 7-18, whereby any of these sequences may be modified by up to three amino acids individually or in combination as long as they remain refractory to CA125 binding.

In some embodiments, functional methods are used to optimize the effect of MES-BSP on killing mesothelin-positive tumor cells in the presence of CD3⁺ lymphocytes using varying genetic linkers. The term “effect” generally refers to a 10% or greater change in target cell killing when agent is incubated alone as compared to CD3⁺ lymphocytes. It may, depending on the antibody and the agent used also refer to a change of at least 5%, 15%, 20%, 25%, 30%, 35%, 40%, 45%, 50%, 55%, 60%, 70%, or 75% as compared to control.

Yet another embodiment comprises methods for screening antibody drug conjugates (ADCs) with various cytotoxins and/or linkers for their pharmacokinetic (PK), pharmacodynamic (PD) or pharmacologic (PL) activity, including cellular internalization. In some embodiments, the ADC is added to cells in vitro and tested for target cell killing. ADCs that have significant killing effect are suitable for therapeutic testing. Again, the term effect may, depending on the antibody and the agent used refer to a change of at least 5%, 10%, 15%, 20%, 25%, 30%, 35%, 40%, 45%, 50%, 55%, 60%, 70%, or 75% as compared to control.

Various terms and terminology (“terms”) relating to aspects of the enclosed description are used throughout the specification and claims of this document. Such terms are to be given their ordinary meaning in the art unless otherwise specifically indicated. Other specifically defined terms are to be construed in a manner consistent with the definitions provided.

As used in this specification and the appended claims, the singular forms of “a,” “an,” and “the” also include plural references unless the content clearly specifically dictates otherwise. As example, reference to “a cell” may include a combination of two or more cells, and the like. Reference to “a probe” may include detector antibody, MES-ADC, MES-BSP or an independent probe to monitor the tumor microenvironment immune status via any analytical method known in the art.

The term “about” as used when referring to quantified values such as an amount, a period of time, and/or the like, is meant to encompass variations of up to ±9% from the specified value, as such variations are appropriate to carry out the disclosed methods. Unless otherwise indicated, all values expressing quantities of reagents, such as molecular weight, molarity, reaction conditions, percentage and so forth used in the specification and claims are to be understood as being quantified in all instances by the term “about.” Accordingly, unless indicated to the contrary, the numerical values as set forth in the following specifications and listed claims are approximations that may vary depending upon the desired properties of the composition agent and/or methods sought to be obtained by the present invention. At the very least, and not as an attempt to limit the scope of the application, each numerical value should at least be valued by the reported significant digits and through ordinary rounding methods known in the art.

The term “antibody” as used is meant in a broad sense and includes immunoglobulin (also referenced as “Ig”) or antibody molecules including polyclonal antibodies (also referenced as pAbs), monoclonal antibodies (also referenced as mAbs) including murine, human, humanized and chimerized mAbs, bispecific antibodies (also referenced as BSP), antibody drug conjugates (also referenced as ADCs), antibody fused immunotoxins and antibody fragments. In general, antibodies are proteins or polypeptide chains that bind to a specific antigen. An antigen is a structure that is specifically recognized by a given antibody. Canonical antibodies comprise heterotetramer glycosylated proteins, composed of two light chains and two heavy chains lined through a complex of disulfide and hydrogen bonds. The term “its disulfide bridge” refers to the disulfide bridge contained within the heavy chain hinge region, as is known in the art. Each heavy chain has a variable domain (variable region) (VH) followed by a number of constant domains (referred to as the Fc domain). Each light chain has a variable domain (VL) and a constant domain; the constant domain of the light chain is aligned with the first constant domain of the heavy chain and the light chain VL is aligned with the variable domain of the heavy chain. Antibody light chains of any species are assigned to one of two distinct types based on their amino acid sequences within their constant domains, namely kappa (κ) and lambda (λ).

The term “single chain” refers to a single chain antibody with the structure known in the art.

An immunoglobulin light chain (LC) or heavy chain (HC) comprises a “framework” region interrupted by three “antigen-binding sites” also referred to as Complementarity Determining Regions (CDRs) based on sequence variability as reported (Wu T T, Kabat E A. J Exp Med 132:211-250, 1970). In general, an antigen-binding site is composed of six CDRs with three located within the VH (CDRH1, CDRH2, CDRH3), and three within the VL (CDRL1, CDRL2, CDRL3) (Kabat E A, et al. 5^{th} Ed. PHS, National Institutes of Health, Bethesda, Md., 1991).

“Specific binding” or “specifically binds” refers to the binding of an antibody or antigen-binding fragment to an antigen (including sequences contained within an antibody itself) with greater affinity than for other antigens. Typically, a specific antibody or antigen-binding fragment binds target antigen with an equilibrium dissociation constant K_{D} of about 5×10⁻⁶ M or less.

An “antibody derivative” or “alternative format” means an antibody, as defined above, that is modified by covalent attachment of another molecule via peptide chemistry (i.e., amidation, etc.), genetic fusion and/or via post translational moieties (i.e., glycosyl, acetyl and/or phosphoryl) not typically associated with the antibody and the like.

The term “antibody dynamic structure” refers to any change in structure that can affect antibody function, CA125 binding or cellular internalization.

The term “monoclonal antibody (mAb)” refers to an antibody that is derived from a single cell clone, including any eukaryotic or prokaryotic cell clone, or a phage clone, and not the method by which it is produced. Thus, the term “monoclonal antibody” is not limited to antibodies produced through hybridoma technology but may also include recombinant methods.

“Fab domain” refers to any antibody sequence N-terminal to the antibody hinge disulfide region which is known in the art.

“Fc domain” refers to any antibody sequence C-terminal to and including the antibody hinge disulfide region which is known in the art.

“Mesothelin” refers to the entire protein or a naturally altered form of the mesothelin protein (GenBank: AAH03512.1).

An “antigen” is an entity to which an antibody or antibody fragment specifically binds. This includes binding to an antibody or protein of interest.

The term “CA125” refers to the gene product produced by MUC16 gene (HGNC: 15582; OMIM: 606154), which is found in soluble and membrane-bound forms. It has been reported to bind to antibodies within the Fab domain and affect the bound antibody's humoral immune function (Kline J B, et al. Oncotarget 8:52045-52060, 2017) as well as ADC uptake.

The term “CA125 refractory” refers to antibodies that have low or no binding to the CA125 protein as measured using any method known in the art.

The term “CD3” and “CD3E” refers to the CD3-epsilon protein expressed on human lymphocytes.

The terms “cancer,” “malignant,” “dysregulated,” and “tumor” are well known in the art and refer to the presence of cells with unregulated cell growth and morphological features different than a normal cell type of similar origin, also referred to as dysregulated cells. Malignant refers to those cancer cells capable of causing morbidity and/or mortality. As used, “cancer and tumor” includes premalignant and malignant types.

As used, the term “soluble” refers to a protein or non-protein agent that is not attached to the cellular membrane of a cell. For example, an agent that is soluble may be shed, secreted or exported from normal or cancerous cells into biological fluids including serum, whole blood, plasma, urine or microfluids of a cell, including tumors.

The “level” of a specified protein or non-protein agent including CA125, as used, refers to the level or levels of the agent as determined using any method known in the art for the measurement of protein and/or non-protein agent levels in vitro or in vivo. Such methods include gel electrophoresis, capillary electrophoresis, high performance liquid chromatography (HPLC), thin layer chromatography (TLC), hyperdiffusion chromatography, fluid or gel precipitation reactions, absorption spectroscopy, colorimetric assays, spectrophotometric assays, flow cytometry, immunodiffusion (single or double), solution phase assay, immunoelectrophoresis, Western blotting, radioimmunoassay (RIA), enzyme-linked immunosorbent assays (ELISAs), immunofluorescent assays, fluorescence resonance energy transfer (FRET), Förster resonance energy transfer, electrochemiluminescence immunoassay, and the like. In one embodiment, the level of CA125 is determined using probe-based techniques, as described in more detail.

The term “humoral immunosuppression or humoral immunesuppression” refers to any antibody, antibody fragment, bispecific antibody (BSP) or antibody drug conjugate (ADC) that is directly bound by CA125 and whose dynamic structure is altered. It has been reported that CA125, which is produced by malignant cells such as ovarian carcinoma and mesothelioma (Nicolaides N C, et al. Cancer Biol Ther 19:622-630, 2018) as well as induced by lymphomas from normal surrounding epithelial cells (Sanusi et al. Perit Dial Int. 21:495-500, 2001), can bind certain antibodies and alter their dynamic structure thus affecting their biological activities including ADCC, CDC, opsonization, internalization and/or PK, PD and PL profiles.

The term “antibody drug conjugate (ADC)” refers to any antibody that is conjugated or fused to a chemical, polypeptide, nucleic acid or radionuclide that has toxic activity to cells.

The term “cleavable linker” means a chemical or amino acid linker that can be cleaved extracellularly or intracellularly by any common mechanism, such as but not limited to enzyme or protease digestion, acid degradation, pH, chemical reduction, chemical oxidation, hydrolysis, etc.

The term “non-cleavable linker” means a chemical or amino acid linker that is not generally cleaved extracellularly by common mechanisms, such as but not limited to enzyme or protease digestion, chemical reduction, chemical oxidation, hydrolysis, etc.

The term “enzymatic cleavable linker” and “enzymatic non-cleavable linker” means linkers that are cleavable or non-cleavable by enzymes or proteases.

The term “bispecific antibody (BSP)” refers to any antibody that can bind two or more different antigens. A BSP can comprise at least but not limited to two full length antibodies, a full length antibody and a single chain antibody, or two single chain antibodies, where each one binds to different antigens or different epitopes on the same antigen.

The term “canonical antibody” refers to an immunoglobulin light chain linked to an immunoglobulin heavy chain, whereby the antibody can specifically recognize a said antigen. The canonical antibody may be fused to another antibody that is capable of specifically recognizing a second antigen.

The term “immunosuppressed microenvironment,” immune-suppressed microenvironment,” “immunosuppressed tumor microenvironment,” immune-suppressed tumor microenvironment” refers to tumors that produce or express factors that suppress cellular or humoral immune functions and activities, such as but not limited to PDL1 or CA125, respectively.

The term “immunocompetent microenvironment,” immune-competent microenvironment,” “immunocompetent tumor microenvironment,” immune-competent tumor microenvironment,” “immune-proficient,” “immunoproficient,” “immune-proficient” refers to tumors that do not produce or express factors that suppress cellular or humoral immune functions or activities.

“Immune or immunomicroenvironment status,” “microenvironment immune status” refers to determination if tumor is immunocompetent or immunosuppressed. The term also refers to tumors that produce immunosuppressive proteins, where tumors producing such proteins are considered to have an immunosuppressed microenvironment.

The term “antibody dependent cellular cytotoxicity (ADCC)” refers to an in vitro or in vivo process where an antibody can bind to an antigen on the surface of a cell then engage with immune-effector cells via sequences within the antibody's Fc domain that in turn results in their release of toxins that can kill bound cell.

The term “complement dependent cytotoxicity (CDC)” refers to an in vitro or in vivo process where an antibody can bind to an antigen on the surface of an eukaryotic or prokaryotic cell then engage with the C1q protein via sequences within the antibody's Fc domain that in turn results in initiation of classical complement cascade that can kill the bound cell.

The term “internalization” refers to a process where an antibody, antibody fragment or ADC can bind to an antigen on a surface of a cell then internalize via mechanisms known to those skilled in the art.

The term “pharmacokinetic (PK)” refers to the time that an antibody maintains its steady-state concentration when administered to a subject.

The term “pharmacodynamic (PD)” refers to the study of the biochemical and physiological effects of an antibody-based drug and its mechanisms of action(s), including the correlation of their actions and effects with their biochemical structure when administered to a subject.

The term “pharmacologic (PL)” refers to the known effect an antibody has on managing or killing a disease cell in vitro or in vivo.

The term “sample” refers to a collection of similar fluids, cells or tissues isolated from a subject, as well as fluids, cells or tissues present within a subject. Fluids may include biological fluids that include liquid solutions contacted with a subject or biological source, including cell and organoid culture medium, urine, salivary, lavage fluids and the like.

The term “control sample,” as used, refers to any clinically or non-clinically relevant control sample, including, for example, a sample from a healthy subject not afflicted with a particular cancer type or a cell that is different from its parental cell.

The term “control level” refers to an accepted or pre-determined level of a protein or non-protein agent that is used to compare with the level of the same agent in a sample derived from a subject or used in in vitro assays.

As used, “a difference” between signal of a therapeutic antibody versus control is generally any difference that can be statistically determined using statistical methods commonly used in the art and at a minimum a difference of 10% or greater as compared to control. It may, depending on the antibody and the probes used also refer to a change of at least 5%, 15%, 20%, 25%, 30%, 35%, 40%, 45%, 50%, 55%, 60%, 70%, or 75%.

The term “inhibit” or “inhibition of” means to reduce by a statistically measurable amount, or to prevent entirely.

The term “functional,” in the context of an antibody, antibody containing moiety (i.e. BSP, ADC, etc.) to be used in accordance with the methods described, indicates that the antibody is capable of binding to antigen or CA125, respectively, and/or is able to bind to and kill target cells in vitro or in vivo.

The term “target cell” refers to a eukaryotic or prokaryotic cell or population of cells that express antigen for a specific antibody or antibody-containing moiety.

The term “therapeutic window” means efficacy of a drug to suppress tumor growth within a manageable tolerated toxicity dosage.

The term “pharmaceutically acceptable” refers to a substance that is acceptable to administer to a patient from a pharmacological as well as toxicological aspect and is manufactured using approaches known in the art. These include agents approved by a regulatory agency of the Federal or a state government or listed in the U.S. Pharmacopeia or other generally recognized pharmacopeia for use in animals and humans. The term “pharmaceutically compatible ingredient” refers to a pharmaceutically acceptable diluent, adjuvant, excipient or matrix vehicle with which an anti-cancer agent is administered. “Pharmaceutically acceptable carrier” refers to a matrix that does not interfere with the effectiveness of the biological activity of the active ingredient(s) and is nontoxic to the host.

The terms “effective amount” and “therapeutically effective” are used interchangeably and, in the context of administering a pharmaceutical agent at an amount that is sufficient to produce an enhanced clinical outcome in a patient. An effective amount of an agent is administered according to the methods described here in an “effective regimen.” The term “effective regimen” refers to a combination of amount of the agent and dosage frequency adequate to accomplish an enhanced clinical outcome for a patient with a particular cancer. Enhanced efficacy is an improved clinical outcome when a patient is administered an agent that is capable of overcoming morbidity better than a parental compound or an agent that can enhance the clinical outcome of an effective regimen.

The terms “patient” and “subject” are used interchangeably to refer to humans and other non-human animals, including veterinary subjects, that receive a therapeutic agent treatment. The term “non-human animal” includes all vertebrates. In one embodiment, the subject is a human.

“Therapeutic agents” are typically substantially free from undesired contaminants. This means that an agent is typically at least about 50% w/w (weight/weight) pure as well as substantially free from interfering proteins and contaminants.

The term “immune-effector cell” refers to any cell including but not limited to NK, myeloid, monocytes, or dendritic cells that may confer antibody dependent cellular cytotoxicity (ADCC) or phagocytosis (opsonization) upon binding to antibody-bound target cell. Cells may be purified or present in mixture in the form of peripheral blood mononuclear cells (PBMCs).

The term “dysregulated cell” refers to any cell that is deemed abnormal to parental cells. These include transformed cells, malignant cells, virally infected cells, autonomously growing cells via autoregulation, or prokaryotic pathogens.

The term “humoral response” refers to ADCC, CDC, opsonization or internalization of antibody into target cells by test antibody.

The term “agent” refers to anti-mesothelin ADC or BSP.

The term “significant(ly)” refers to statistical results where the P value as determined by any number of programs including the Student's T-Test is less than 0.05.

Composition of Detector Antibody, Therapeutic MES-ADC and MES-BSP; Kits and Methods for Treating Patients with Mesothelin Expressing Cancers

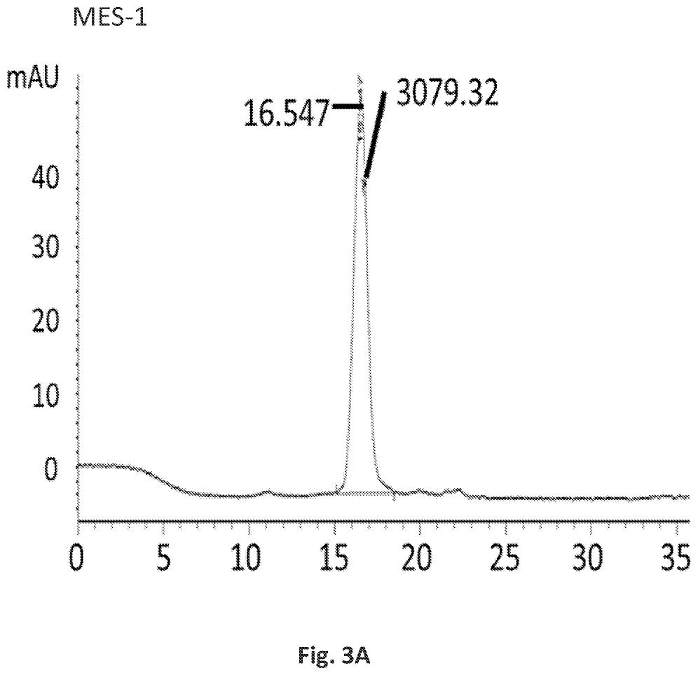

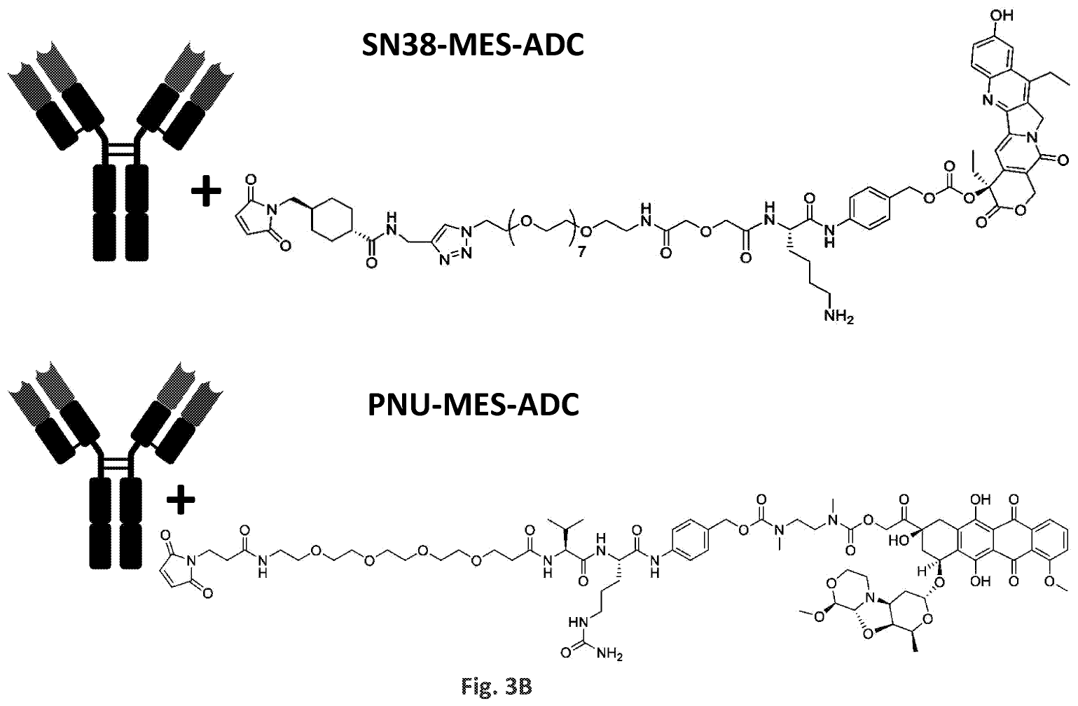

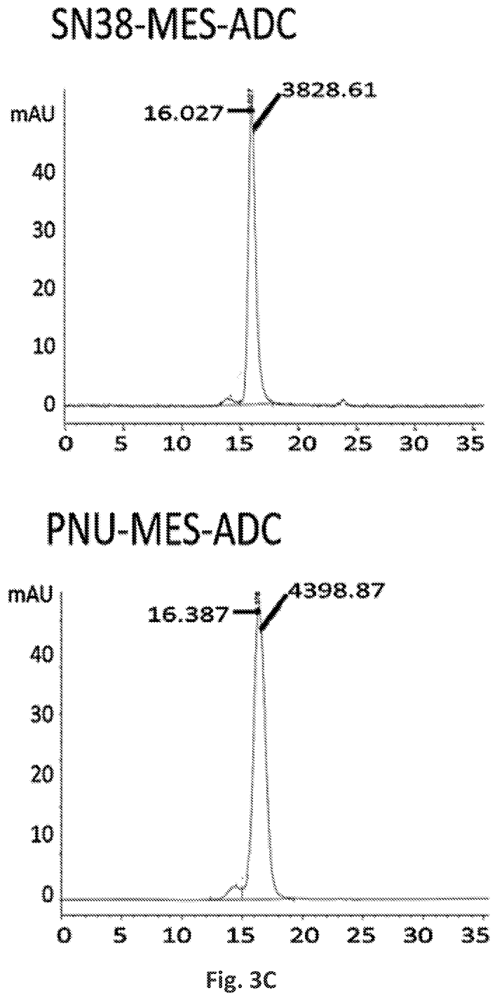

Provided here are compositions of MES-ADC and MES-BSP (both referred to as agents) and rMES-1 (referred to as detector antibody), kits, and methods for identifying such agents that can effectively suppress mesothelin-positive cancers irrespective of their microenvironment immune status. In some embodiments of the methods for identifying optimal MES-ADC and MES-BSP described here, the method involves identifying antibody components of ADCs and/or BSPs that are CA125 refractory and can avoid any of its negative tumor cell killing activities (i.e., tumor uptake of ADC, immune response of bispecific antibody, etc.). In other embodiments a CA125 refractory MES-ADC is composed of two or more cytotoxins linked via a cleavable or non-cleavable linker and testing for the ability of MES-ADC to have significantly improved cytotoxicity against mesothelin expressing immunosuppressed target cells and also effective against immunoproficient target cells. In some embodiments, the MES-ADC cytotoxin is a topoisomerase inhibitor of the SN38 or PNU159682 class. In another embodiment, the MES-ADC comprises the MES-1 antibody conjugated to SN38 via linker MAC glucuronide phenol or PEG8-triazole-PABC-peptide-mc at a drug:antibody ratio (DAR) of two to six. In yet, another embodiment, the MES-ADC comprises the MES-1 antibody conjugated to PNU159682 via linker MA-PEG4-VC-PAB-DMAE at a DAR of two to six. Examples are schematically shown in FIG. 3B. Kits are composed of the MES-ADC that are able to identify optimal ADC format (cytotoxin and linker) by employing ADC killing assays against immunosuppressed and immunoproficient mesothelin expressing tumor types via screening assays used in the art. In addition kits are composed of optimal MES-ADC plus rMES-1 detector antibody, both of which can bind mesothelin in the presence or absence of CA125 to identify patients with mesothelin expressing cancers for treatment with MES-ADC irrespective of tumor microenvironment immune status.

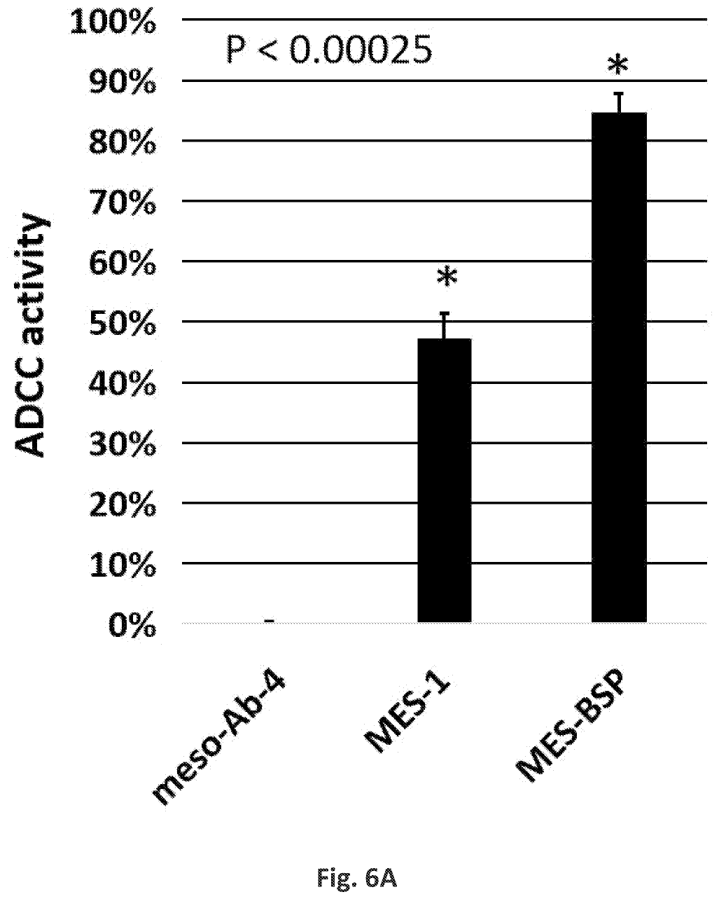

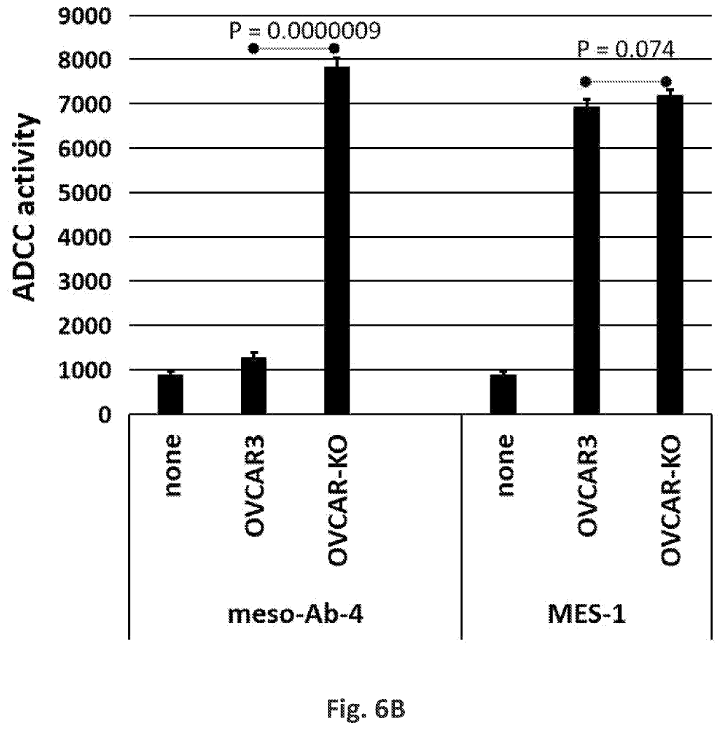

Another embodiment is the method for identifying optimal MES-BSP. The method involves generating optimized MES-BSP, where the MES-BSP light chain contains the amino acid listed in SEQ ID NO: 1 or the heavy chain with amino acids listed in SEQ ID NO: 2 linked to an anti-CD3 single chain antibody (SEQ ID NO: 6) at the N-terminus. In other embodiments, the MES-BSP comprises the MES-1 light chain fused to the anti-CD3 single chain via a genetically linked spacer. While the spacer may consist of any combination and length of natural or modified amino acids, one optional linkage between the MES-1 light chain and CD3 single chain are through genetically encoded linker unit(s) “GGGGS (SEQ ID NO: 20.” Linkage can be by one or multiple units. Optimal spacer units can be determined using functional killing assays of immunosuppressed and immunoproficient mesothelin-expressing target cells using assays commonly employed in the art. Examples of screening are discussed in Example 3 and results shown in FIG. 6. In another embodiment, kits are composed of optimal MES-BSP plus rMES-1 detector antibody to identify patients with mesothelin-expressing cancers for treatment with MES-BSP irrespective of tumor microenvironment immune status.

In the methods for identifying optimally active MES-ADC or MES-BSP agents, the antibody is added to a culture of mesothelin-expressing target cells in which target cells naturally or recombinantly express an immunosuppressive protein. Cultures comparing response to MES-ADC or MES-BSP treatment vs control-treated cells are monitored for target cell viability using standard killing assays. A change in at least 10% is typically considered as being a meaningful effect. Depending on the agent and the assay employed, a meaningful effect also may be defined as a change of at least 5%, 15%, 20%, 25%, 30%, 35%, 40%, 45%, 50%, 55%, 60%, 70%, or 75%.

In some methods for identifying optimally active MES-ADC or MES-BSP, the agent is added to a culture of mesothelin expressing target cells in which target cells naturally express CA125 or the soluble CA125 protein is exogenously added. Cultures comparing response of those treated with MES-ADC or MES-BSP plus CA125 vs those treated with controls or no CA125 are monitored for target cell viability using standard killing assays. A change of at least 10% enhanced killing is typically considered as being a meaningful effect on ADCC, CDC and/or ADC target cell killing. Depending on the agent and the assay employed, a meaningful effect also may be defined as a change of at least 5%, 15%, 20%, 25%, 30%, 35%, 40%, 45%, 50%, 55%, 60%, 70%, or 75%.

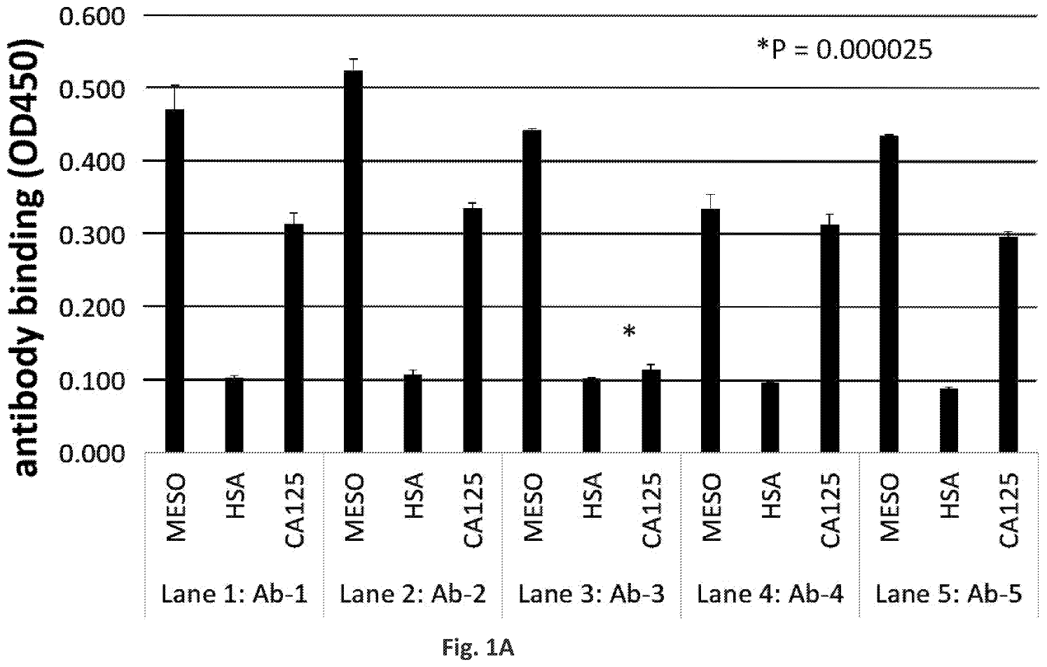

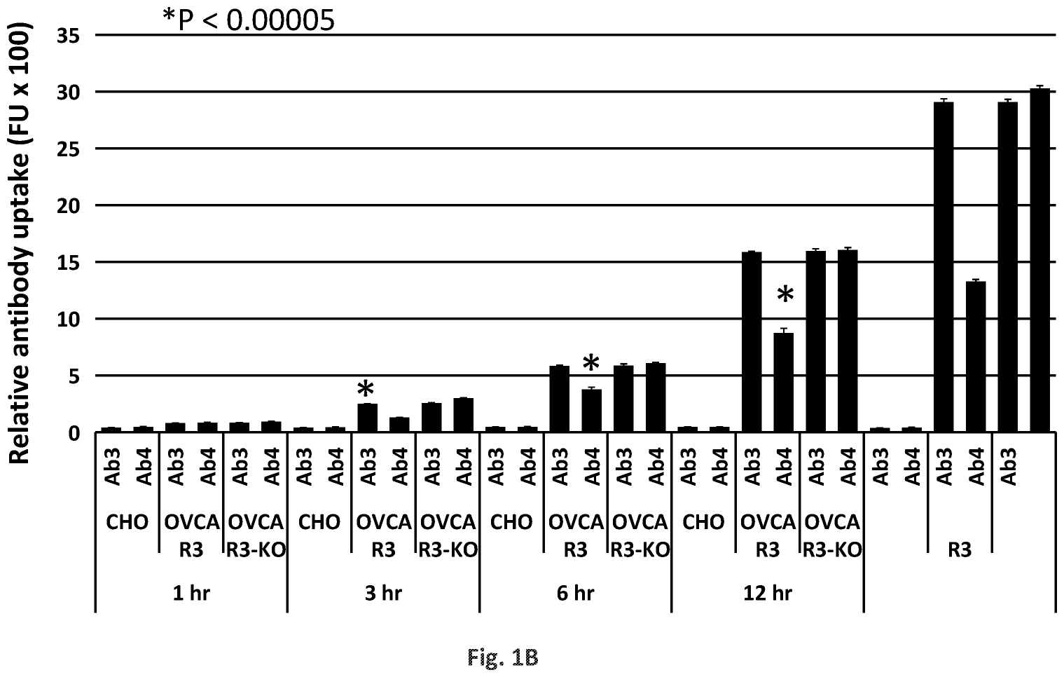

Also provided here are methods of treating a cancer subject with a MES-ADC or MES-BSP agent. For example, a patient may have a mesothelin-expressing cancer such as but not limited to mesothelioma, colorectal, lung, ovarian, pancreatic, cholangio, or endometrial carcinoma. Several anti-mesothelin antibodies have been reported to be bound by CA125 (refer to FIG. 1) that may perturb their internalization as an ADC or suppress their immune killing effect as a BSP making the use of a MES-ADC or MES-BSP that is not bound or affected by CA125 a desirable entity.

In some embodiments of the methods of treating a subject with a MES-ADC or MES-BSP agent, a patient with cancer that expresses an immunosuppressive protein such as CA125, therefore making its tumor microenvironment immunosuppressed, may be treated with a MES-ADC or MES-BSP agent alone or in combination with standard-of-care therapy. In some embodiments of the methods of treating a subject with an immunosuppressed microenvironment, mesothelin-expressing cancers that are known to express CA125 are described here. A MES-ADC or MES-BSP agent is administered to the subject, where the subject has a baseline CA125 level that is above the normal range. In some embodiments of the methods of treating a subject with CA125-expressing cancer described here, the method involves administering the MES-ADC or MES-BSP agent alone. In yet another embodiment, the MES-ADC or MES-BSP agent is administered in combination with chemotherapy. The chemotherapy may be any chemotherapeutic or biological agent considered standard-of-care at the time when the subject is treated. In the methods of treatment described here, CA125 expression levels may be determined by any means known in the art and defined as within or above the normal range in the art.

In other embodiments, a patient is identified for having a mesothelin-expressing cancer using the rMES-1 detector antibody and those positively bound by rMES are treated with MES-ADC or MES-BSP agent without determining the tumor microenvironment immune status as the MES-ADC or MES-BSP is effective in both immunosuppressed and immunoproficient microenvironments. In some embodiments of the methods of treating a subject with mesothelin-expressing cancer described here, the method involves administering the MES-ADC or MES-BSP agent alone. In yet another embodiment, the MES-ADC or MES-BSP agent is administered in combination with chemotherapy. The chemotherapy may be any chemotherapeutic or biological agent considered standard-of-care at the time when the subject is treated.

In some embodiments of the methods of treatment described here, exemplary cancers known to express mesothelin include but are not limited to mesothelioma, lung, colorectal, ovarian, endometrial, choliangio, gastric, breast and pancreatic cancers, many of which have been reported to produce CA125.

The present methods can be combined with other means of treatment such as surgery (e.g., debulking surgery), radiation, targeted therapy, chemotherapy, immunotherapy, use of growth factor inhibitors, or anti-angiogenesis factors. An MES-ADC or MES-BSP agent can be administered concurrently to a patient undergoing surgery, chemotherapy or radiation therapy treatments. Alternatively, a patient can undergo surgery, chemotherapy or radiation therapy prior to or subsequent to administration of the MES-ADC or MES-BSP agent by at least an hour and up to several months, prior or subsequent to administration of standard of care therapy. Some embodiments of the methods of treatment provided here involve administration of a therapeutically effective amount of a platinum-based chemotherapy and/or a folate antimetabolite and/or a PARP inhibitor, with or without an antibody to a tumor-specific antigen or immune checkpoint protein, to the subject in addition to the MES-ADC or MES-BSP agent.

In some embodiments of the methods of treatment described here, the subject may have received first-line surgical resection of the tumor, first-line platinum-based therapy, first-line folate antimetabolite-based therapy, first-line platinum and folate antimetabolite-based therapy, PARP inhibitor and/or an immune checkpoint inhibitor for treatment of the cancer prior to administering an MES-ADC or MES-BSP agent.

Administration of the therapeutic agents (including the MES-ADC or MES-BSP agent, the folate antimetabolite, the platinum-based chemotherapy, PARP inhibitor and/or immune checkpoint inhibitor) in accordance with the methods of treatment described here may be by any means known in the art.

In yet another embodiment, a MES-ADC or MES-BSP agent may be used that comprises the CDR sequences contained within SEQ ID NOs: 7-12, numbered according to IMGT® (the international ImMunoGeneTics information System®) outside of a canonical antibody format. Administration of these modified MES-ADC or MES-BSP agents can be prior to, concomitant with or after administration of any additional standard-of-care treatment. Treatment can include surgery as well as treatment with current standards-of-care used at the time of treatment.

Various delivery systems can be used to administer the therapeutic agents (including the MES-ADC or MES-BSP agent) including intradermal, intramuscular, intraperitoneal, intravenous, subcutaneous, intranasal, epidural, and oral routes as deemed necessary. The agents can be administered, for example by infusion or bolus injection, by absorption through epithelial or mucocutaneous linings (e.g., oral mucosa, rectal and intestinal mucosa, and the like) via systemic or local approaches.

The therapeutic agents can be administered by injection via syringe, catheter, suppository, or any implantable matrix or device.

The therapeutic agents and pharmaceutical compositions thereof for use as described here may be administered orally in any acceptable dosage form such as capsules, tablets, aqueous suspensions, solutions or the like.

Suitable methods of administration of the therapeutic agents, include but are not limited to, intravenous injection and intraperitoneal administration at a final concentration suitable for effective therapy.

The MES-ADC or MES-BSP agent in combination with other drugs can be administered as pharmaceutical compositions comprising a therapeutically or prophylactically effective amount of the therapeutic agent(s) and one or more pharmaceutically acceptable or compatible ingredients.

The amount of the therapeutic agent that is effective in the treatment or prophylaxis of a cancer can be determined by standard clinical techniques. In addition, in vitro assays may optionally be employed to help identify optimal dosage ranges required for the MES-ADC or MES-BSP agent. Effective doses may be extrapolated from dose-response curves of MES-ADC or MES-BSP agents derived from in vitro cell based assays, animal models or other non-human test systems.

For example, toxicity and therapeutic efficacy of the agents can be determined in cell cultures or experimental animals by standard pharmaceutical procedures for determining the LD₅₀ (the dose lethal to 50% of the population) and the ED₅₀ (the dose therapeutically effective in 50% of the population) values. The dose ratio between toxic and therapeutic effects is the therapeutic index or window and it can be expressed as the ratio LD₅₀/ED₅₀. Agents that exhibit large therapeutic indices are suitable. When an agent exhibits toxic side effects, a delivery system that targets the agent to the site of affected tissue can be used to minimize potential damage to non-mesothelin-expressing cells and, thereby, reduce side effects. Alternatively, formulations such as but not limited to liposomal encapsulation may be used to improve the therapeutic index if required.

The dosing and dosage schedule may vary depending on the active drug concentration, which may depend on the needs of the subject.

Another embodiment labels a rMES-1 detector antibody for detection of mesothelin-expressing cells in CA125 expressing or non-expressing tumors for diagnostic applications to detect and/or monitor the status of a mesothelin-expressing tumor cells in vitro or in situ during or after treatment with MES-ADC or MES-BSP agent. Labeling can be any method used to label antibodies for diagnostic monitoring known in the art.

### Composition of Kits to Optimize Activity of MES-ADC and MES-BSP

Further provided here are kits for making optimized MES-ADC and MES-BSP agents suitable for killing immunosuppressed and immunoproficient mesothelin expressing tumors.

Kits may include a MES-ADC agent containing a cytotoxin linked to an antibody comprised of SEQ ID NOs: 1 and 2 that is able to kill two or more types of mesothelin expressing cells or tumors irrespective microenvironment immune status, where the cytotoxin has topoisomerase inhibitory activity with an IC50 of ≤100 μM.

Kits may include a MES-BSP agent containing an antibody or antibody fragment with SEQ ID NOs: 7-12 linked to an anti-CD3 single chain (SEQ ID NO: 6) that is able to kill immunosuppressed and immunoproficient mesothelin expressing cells or tumors, where the linkage between anti-mesothelin antibody and anti-CD3 antibody is through an optimized spacer that result in killing mesothelin expressing cells at an IC50 of ≤100 μg/mL.

The above disclosures generally describe the present invention. All references disclosed here are expressly incorporated by reference. A more complete understanding can be obtained by reference to the following specific examples which are provided here for purposes of illustration only, and are not intended to limit the scope of the invention.

### Example 1—Screening for Anti-Mesothelin Antibodies Naturally Refractory to Immunosuppressive Tumor-Produced Proteins

Several studies have reported that antibodies that are bound by CA125 are negatively affected in eliciting humoral mediated immune killing as well as ADC-mediated killing of target cells (Kline J B, et. al. OncoTarget 8:52045-52060, 2017; Kline J B, et al. Eur J Immunol 48:1872-1882, 2018; Kline J B, et al. J Clin Oncol 5:15, 2018; Nicolaides N C, et al. Cancer Biol Ther 13:1-22, 2018; Nicolaides et. al. USPTO application 1698444). In an attempt to identify anti-mesothelin antibodies that naturally are not bound by CA125, we obtained a number of anti-mesothelin antibodies from academic and commercial sources (National Cancer Institute, Creative Biolabs, Novus, etc.) and tested them for CA125 binding. FIG. 1 shows representative results of an enzyme linked immune assay (ELISA) to screen for the binding of different anti-mesothelin antibodies by CA125. Recombinant mesothelin was used as a positive control and human serum albumin (HSA) was used as negative control. Briefly, 96-well plates were coated with 50 μL/well of 15 KU/mL human CA125 protein, 1 μg/mL of mesothelin or 1 μg/mL of HSA in 0.05M carbonate buffer, pH 9.5 overnight at 4° C. The next day, plates were washed with 125 uL of 0.05M phosphate buffer, pH7.2 three times then blocked in 0.05M phosphate buffer with 5% bovine serum albumin at room temperature for 1 hour. Wells were then washed with 125 μL of 0.05M phosphate buffer, pH7.2 three times and probed with 2.5 μg/mL of the various anti-mesothelin antibodies (meso-Abl through meso-Ab5). As shown in FIG. 1, several anti-mesothelin antibodies were bound by CA125 while meso-Ab3 (lane 3), which is comprised of the MES-1 antibody (SEQ ID NO:1 and SEQ ID NO:2) unexpectedly did not. All antibodies bound the mesothelin protein at similar values while none bound to the HSA negative control. These results identified MES-1 as a naturally occurring, CA125-refractory binding antibody and suggest the use of such an antibody for treating cancer cells or tumors that produce such immunosuppressive factors. To confirm that CA125 binding differentially impacted internalization of MES-1 (FIG. 1A Ab-3) vs an anti-mesothelin antibody that is bound by CA125 Ab-4 (FIG. 1A), we conducted internalization assays using mesothelin expressing OVCAR3 cells that naturally overexpress membrane bound CA125 (Kline J B, et. al. OncoTarget 8:52045-52060, 2017) and an OVCAR3 CA125 knockdown line via shRNA that was created using a similar strategy as that reported by Kline et al. to generate isogenic cells (referred to as OVCAR-KO) for comparison. Both antibodies were fluorescently labeled using the pH sensitive pHrodo fluorescent dye system following the manufacturer's protocol (ThermoFisher, thermofisher.com/adcdiscovery). Antibodies were then incubated with OVCAR3 or OVCAR-KO cells in replicas in 96-well microplates over a 24 hour period and quantified for cellular uptake via fluorescence using a Varioskan™ plate reader (ThermoFisher). As shown in FIG. 1B, the MES-1 Ab (Ab-3) had efficient uptake in both CA125 expressing OVCAR3 and the CA125 knockdown OVCAR-KO cells in contrast to Ab-4, supporting the unexpected finding that the sequences encoding the MES-1 antibody are naturally CA125 refractory, making it a qualified antibody ADC component to treat CA125 expressing tumor cells using the methods taught herein.

### Example 2—Generation of Anti-Mesothelin Antibody Drug Conjugates (ADC) Capable of Killing Immunosuppressed and Immunoproficient Mesothelin Expressing Cancer Cells

To determine if MES-1 was refractory to the immunosuppressive effects of CA125 and potentially other immunosuppressive proteins produced by mesothelin-expressing tumor cells, we configured MES-1 into an ADC format and tested its effectiveness in killing CA125-expressing, immunosuppressed, mesothelin-expressing cancer cells. Here we provide biochemical analysis of MES-1 and MES-ADC compositions and examples of efficacy of MES-ADCs in various formats required to effectively kill immunosuppressed and immunoproficient mesothelin-expressing tumor cells. We provide the use of a CA125 non-binding, anti-mesothelin antibody (SEQ ID NO: 1 and SEQ ID NO: 2) linked to a certain payload and certain linker type to maximize killing of immunosuppressed target cells. As discussed above, Nicolaides et. al. (Cancer Biol Ther 19:622-630, 2018; Nicolaides et. al. USPTO application Ser. No. 16/981,444) have reported that CA125 binding to the anti-mesothelin antibody amatuximab results in suppressed humoral immune function as well as reduced ADC killing by ADCs to any target antigen that are bound by CA125 due to reduced tumor uptake as compared to those that are not bound by CA125. To determine the maximal efficacy that a MES-1 antibody in ADC format may have against tumors with immunoproficient and immunosuppressive microenvironments, we tested several MES-1 based ADCs linked to a variety of cytotoxic payloads and a variety of linker combinations in vitro and then in human tumor xenografts. To create these ADCs, we first had to generate recombinant MES-1 in a system that would produce homogenous MES-1 protein. Recombinant Chinese hamster ovarian (CHO) cells were employed for recombinant MES-1 production as this system has been previously used to produce high quantities of homogenous antibodies. cDNAs encoding the MES-1 light chain (SEQ ID NO: 4) and the MES-1 heavy chain (SEQ ID NO: 5) were synthesized by polymerase chain reaction (PCR) and PCR fragments were cloned into the pXC vector that has two CMV driven expression cloning cassettes and the glutamine synthetase (GS) gene cassette, referred here as pNAV0047. To generate stable recombinant fusion protein production cell lines, CHOK1SV-GSKO cells containing a knocked out endogenous GS gene were cultured at 6×10⁵ cell/mL in CD-CHO media (Irving Scientific) plus 6 mM L-Glutamine overnight at 37° C. in 5% CO₂. The next day, 2.0×10⁷ cells were resuspended with 20 μg of expression plasmid pNAV0047 in a total volume of 700 μL in CD-CHO media and transferred to a 0.4 cm electroporation cuvette and electroporated at 300 V/900 μF using a BioRad GenePulser II. Cells were immediately transferred to a flask containing 30 mL of CD-CHO media plus 6 mM L-Glutamine and incubated overnight at 37° C. in 5% CO₂ in a shaking platform incubator. The following day, cells were harvested and resuspended in 30 mL CD-CHO/SP4 media containing 50 μM MSX to select for high titer producing clones over a two week selection period. The selected pool was then subcloned by limiting dilution and established clones tested for recombinant MES-1 antibody production. Productive subclones (expressing greater than 1 mg/mL) were expanded and analyzed for antibody production and quality (target antigen binding by ELISA and protein homogeneity by SEC-HPLC). The best quality clone was then expanded and antibody was purified from culture media using Protein A column affinity chromatography following dialysis in PBS buffer. Antibody was quantified and analyzed for homogeneity via size exclusion chromatography (SEC-HPLC) and antigen binding. As shown in FIG. 3A, the MES-1 production line was able to generate high quality, homogenous antibody and was used for ADC generation.

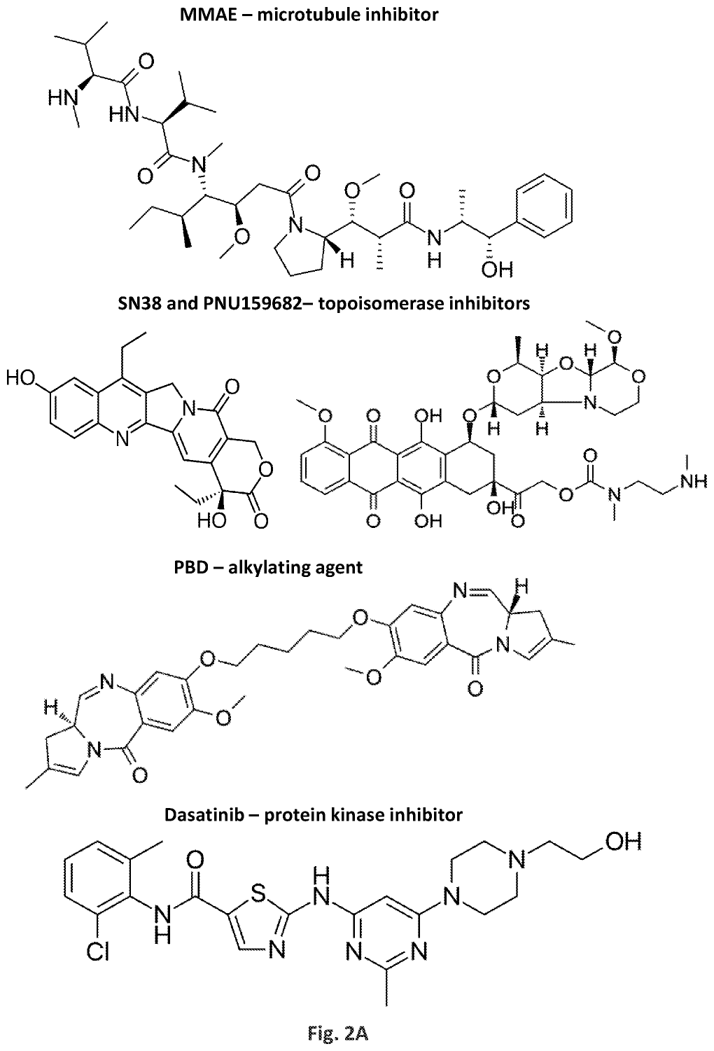

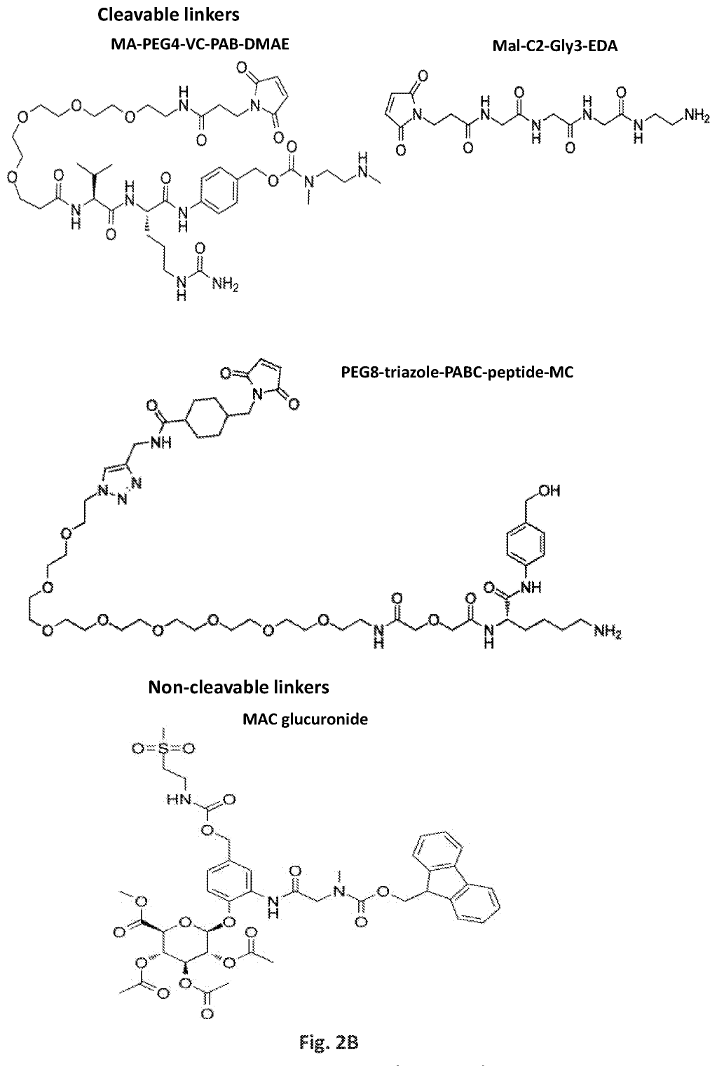

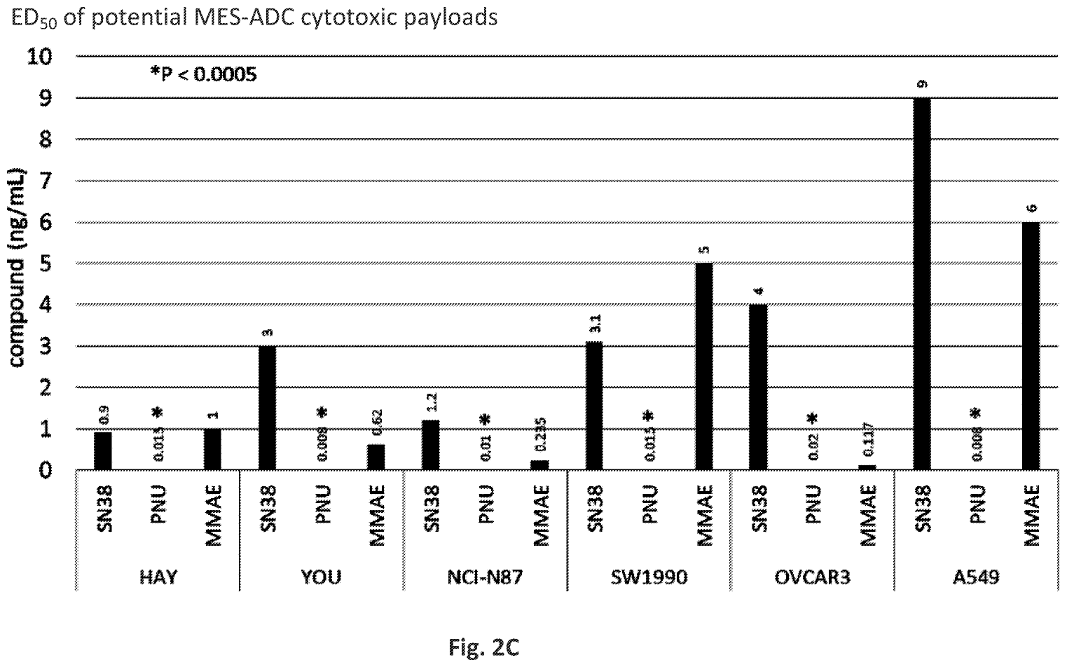

Here we describe the screening for effective cytotoxic-linker combinations that work equally effectively against immunosuppressed and immunoproficient target cancer cells. To identify best candidates to meet this criteria, we first tested different classes of cytotoxins including DNA alkylating agents, microtubule inhibitors, and topoisomerase inhibitors (FIG. 2A). In addition, we also employed different linker combinations (cleavable vs non-cleavable) to determine if they impacted efficacy (FIG. 2B). To test their efficacy, we employed several mesothelin expressing tumor cell lines, a subset of which also co-express the immunosuppressive CA125 protein. These cell lines are listed in Table 1.

TABLE 1
Cell lines tested for MES-ADC killing

| | | Mesothelin | CA125 |
|---|---|---|---|
| cell line | Description | expression | expression |
| NCI-N87 | Human gastric cancer | Yes | Yes |
| A549 | Human lung cancer | No | No |
| HAY | Human mesothelioma | Yes | No |
| YOU | Human mesothelioma | Yes | Yes |
| OVCAR3 | Human ovarian cancer | Yes | Yes |
| SW1990 | Human pancreatic cancer | Yes | Yes |
| CHO | Chinese hamster ovary | No | No |
| CHO-MESO | CHO expressing human | Yes | No |
| | mesothelin | | |

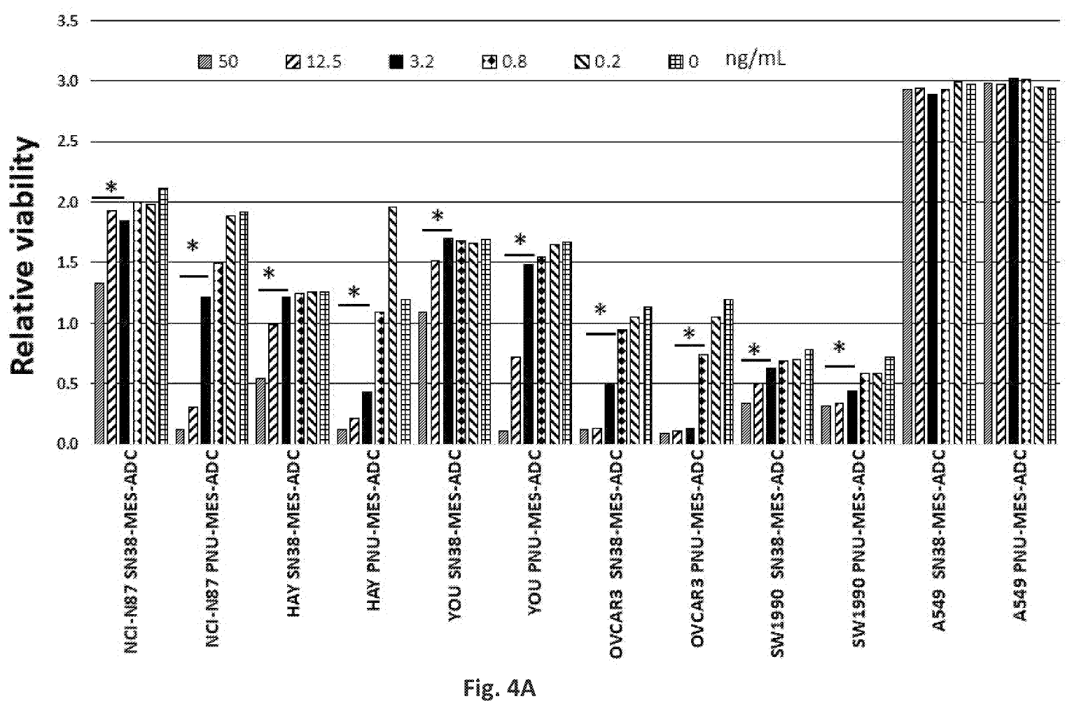

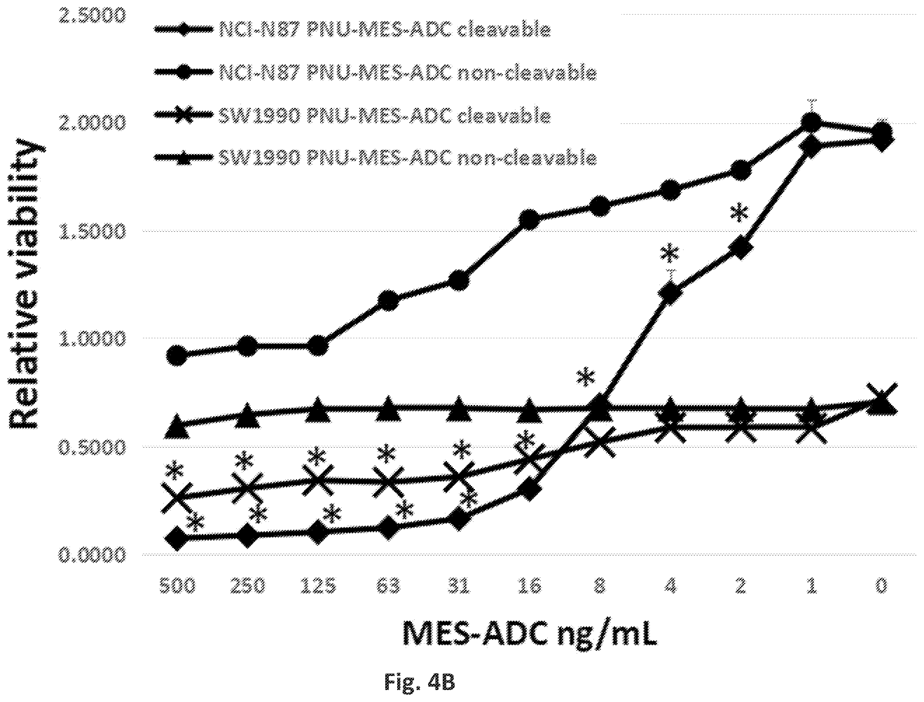

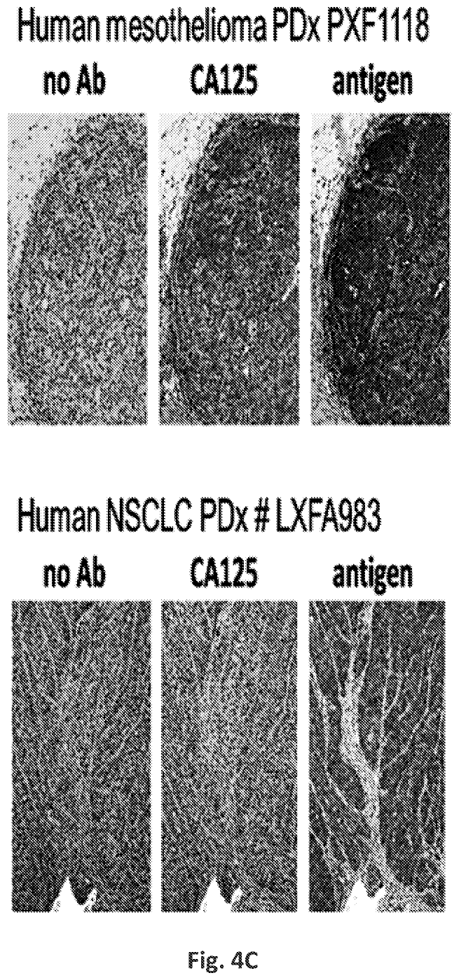

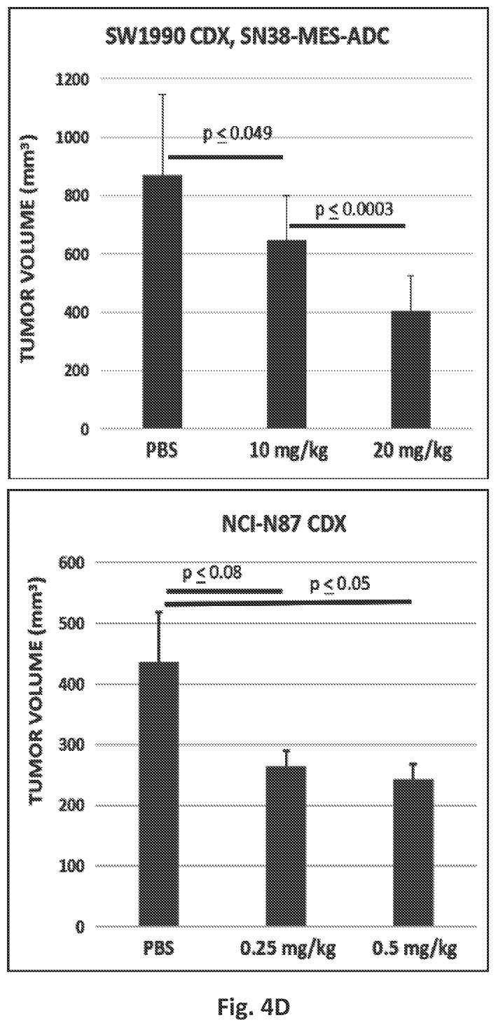

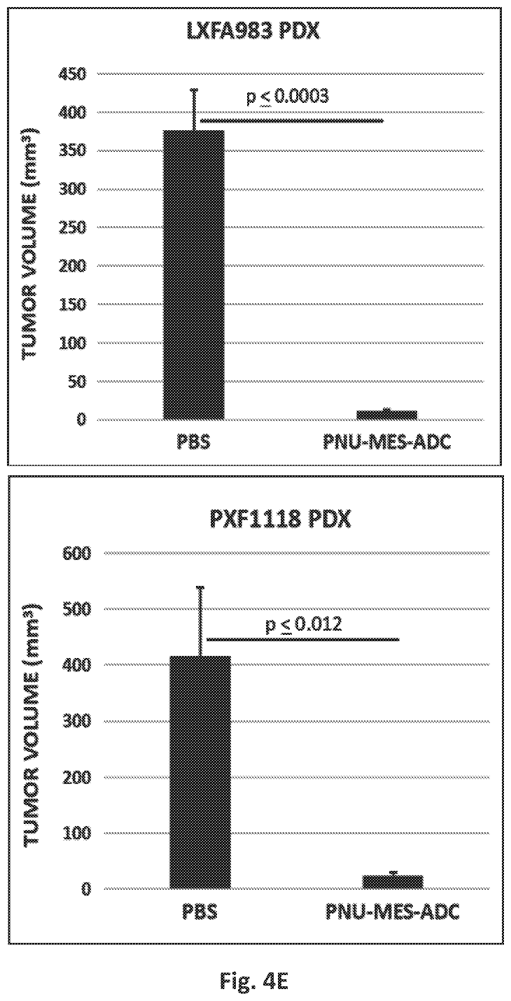

The most potent cytotoxins identified in our screens were the auristatin microtubule inhibitor MMAE (average EC₅₀ 1.39 ng/mL) (Li C, et. al. MAbs 12:1699768, 2020), the two topoisomerase inhibitors SN38 (average EC₅₀ 2.44 ng/mL) (Meyer-Losic F, et. al. Clin Cancer Res 14:2145-2155, 2008) and PNU159682 (average EC₅₀ 0.014 ng/mL) (referred here as PNU) (Quintieri L, et. al. Clin Cancer Res 11:1608-1617, 2005; Carlson R H. Oncol Times 38:8-10, 2016) (FIG. 2C). Based on these results we next engineered the lead cytotoxins into ADC formats using standard cleavable linkers and re-tested them against our tumor cell line panel shown in Table 1. While previous reports have shown that drug:antibody ratio (DAR) is an important feature for ADC target cell killing, it is also a parameter that is sometimes difficult to control during manufacturing when using partial reduction and chemical linkage to free cysteines (Farras M, et. al. Mabs. 12:1702262, 2020). A schematic of SN38-MES-ADC and PNU-MES-ADC is provided in FIG. 3B. Many times controlling DAR reproducibility is inherent to the starting antibody structure and purity. As shown in FIG. 3A, the purification of MES-1 antibody from our manufacturing system has enabled us to generate homogeneous starting antibody and when partially denatured routinely yielded ADCs with a DAR of two to four or four to six (FIG. 3C). Cell killing assays of preliminary MES-ADCs using cleavable linkers found that the SN38- and PNU-MES-ADCs showed the most potent targeted killing effect of all mesothelin expressing lines irrespective of CA125 status likely due to unperturbed cellular uptake while lines not expressing mesothelin were unaffected (FIG. 4A). Since the PNU-MES-ADC showed the most robust killing activity, we next tested PNU-MES-ADC in a cleavable (MA-PEG4-VC-PAB-DMAE) and non-enzymatic cleavable format (MC-EDA) and unexpectedly found that the cleavable format was almost 100 fold more potent than the non-enzymatic cleavable format (FIG. 4B).

To determine lead MES-ADC agent potency in vivo, we next tested the therapeutic efficacy of SN38-MES-ADC and the PNU-MES-ADC cleavable and non-enzymatic cleavable formats in mouse xenograft models using mesothelin and CA125 expressing tumor cell lines NCI-N87 and SW1990 as well as patient derived tumor xenografts (PDX) with mesothelin-expressing tumors with and without CA125 expression. To confirm mesothelin and CA125 expression in xenograft and PDX tumors we tested xenograft-derived fragments via immunohistochemistry (IHC) using the rMES-1 detector antibody which contains SEQ ID NOs: 7-12 on a rabbit IgG backbone and a commercial anti-CA125 antibody, respectively (FIG. 4C). Briefly, 5 μM sections of paraffin embedded tumor fragment were sectioned then adhered to glass slides. Sections were deparaffinized and prepared for antigen retrieval in boiling 10 mM sodium citrate pH 6.0 for 10 min, then equilibrated with phosphate buffered saline-0.05% tween-20 (PBS-T). Sections were then quenched for endogenous peroxidase activity using 0.3% peroxidase/methanol for 10 min and blocked for 1 hour in 10% goat serum in PBS-T. Next, slides were rinsed in PBS-T and probed for mesothelin via rMES-1 or a rabbit anti-CA125 (Novus) using 3 mg/mL of each primary antibody diluted in blocking buffer for 1.5 hours followed by washing, secondary blocking for 1 hour and probing with 5 μg/mL of an anti-rabbit-horseradish peroxidase (HRP) conjugated secondary antibody for 1 hour. Control slides were incubated with no primary antibody. Slides were washed in PBS-T then exposed using eBioscience™ DAB advanced chromogenic substrate as recommended by the manufacturer (ThermoScientific). Finally, samples were hematoxylin counterstained, cover-slipped and analyzed for antigen expression under light microscopy. Both the NCI-N87 and SW1990 xenograft tumors were found to co-express mesothelin and CA125 (not shown), while non-small cell lung adenocarcinoma PDX #LXFA983 and the mesothelioma PDX #PXF1118 were found to express equal levels of mesothelin and while only PDX #PXF118 showed CA125 positivity (FIG. 4C, top panel). We then used these lines in mouse CDXs (cell line derived xenograft) and PDXs (patient derived xenograft) in vivo assays.

For the SW1990 pancreatic cancer CDX model, 1×10⁷ tumor cells were injected into the flank of multiple athymic nude mice. Mice with established tumors (136-154 mm³) were randomized and treated intravenously with either SN38-MES-ADC on day 8, 9, and 10 post implantation at either 10 or 20 mg/kg, or with PBS. SN38-MES-ADC treatment reduced tumor growth by 25% or 53% at 10 or 20 mg/kg respectively, vs. vehicle-treated group on day 39 and the difference was statistically significant (P≤0.049 at 10 mg/kg; P≤0.0003 at 20 mg/kg) (FIG. 4D, top panel).

For the NCI-N87 gastric cancer CDX model, 1×10⁷ tumor cells were injected into the flank of multiple SCID mice. Mice with established tumors (126 mm³) were randomized and treated intravenously as indicated below. PNU-MES-ADC in its enzymatic cleavable format was dosed on day 1, 8, 15, 25, 32, and 44 at 0.25 or 0.5 mg/kg post randomization. PNU-MES-ADC cleavable format treatment reduced tumor growth by 45% at 0.5 mg/kg and the difference was statistically significant (p≤0.050) (FIG. 4D, bottom panel). In the same NCI-N87 gastric cancer CDX model, mice with established tumors (126 mm³) were randomized and treated intravenously with either SN38-MES-ADC on day 1, 3, 5, 7, 9, 11, 13, 15 post randomization at 20 mg/kg, PNU-MES-ADC non-enzymatic cleavable format (0.625 mg/kg on day 1, 1.25 mg/kg on day 5, 2.5 mg/kg on day 9, 5 mg/kg on day 13), or with PBS. SN38-MES-ADC treatment reduced tumor growth by 48% and the difference was statistically significant (p≤0.011) (not shown). PNU-MES-ADC non-enzymatic cleavable format treatment reduced tumor growth by 36% and the difference was statistically significant (P≤0.033) albeit less effective than the PNU-MES-ADC with the enzymatic cleavable format.

For the LXFA983 NSCLC PDX model, tumor fragments were implanted into the flank of multiple athymic nude mice. Mice with established tumors (128 mm³) were randomized and treated intravenously with either PNU-MES-ADC cleavable format on day 1, 4, 8, 12, and 16 post randomization at 0.625 mg/kg (day 1, 4, and 8) or 0.4 mg/kg (day 12 and 16), or with PBS. PNU-MES-ADC cleavable format induced significant tumor regression that was statistically significant (p≤0.0003) (FIG. 4E, top panel).

For the PXF1118 mesothelin PDX model, tumor fragments were implanted into the flank of multiple athymic nude mice. Mice with established tumors (117-124 mm³) were randomized and treated intravenously with either PNU-MES-ADC cleavable format on day 1, 4, 8, 12, 16, and 20 post randomization at 0.625 mg/kg (day 1, 4, and 8) or 0.4 mg/kg (day 12, 16, and 20), or with PBS. PNU-MES-ADC cleavable format induced significant tumor regression that was statistically significant (p≤0.012) (FIG. 4E, bottom panel).

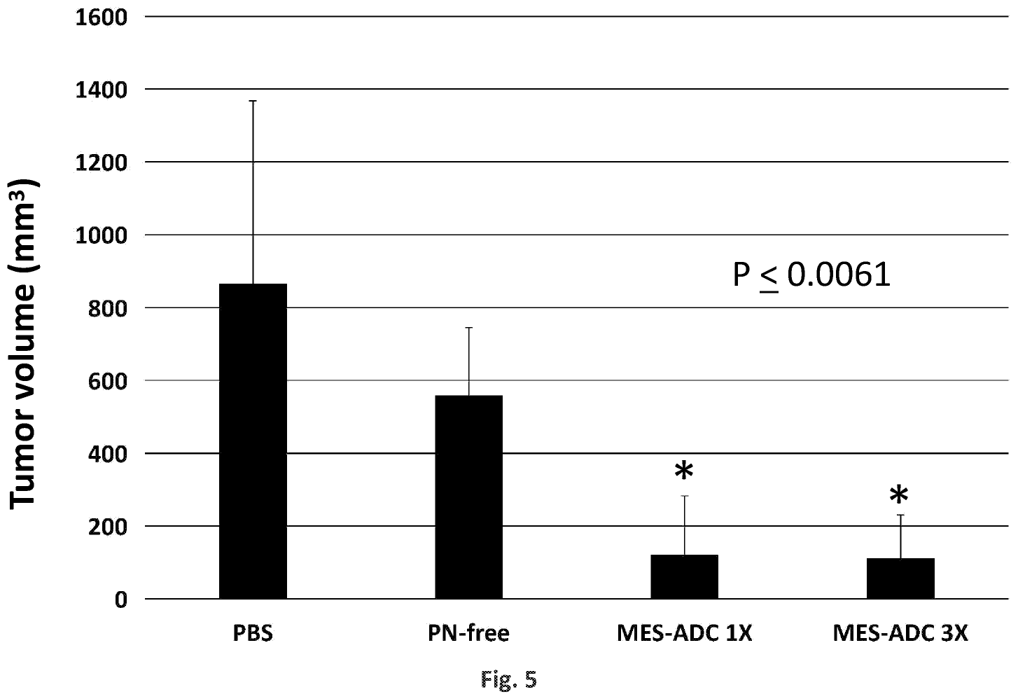

Repeat in vivo experiments were conducted using the CA125 expressing PXF1118 PDX line in model designs as described above to further determine the efficacy of reduced and single PNU-MES-ADC dosing. Briefly, athymic nude mice were prepared as above and grouped into 4 groups of six mice each once established tumors were confirmed. Mice were then treated on day 1, 8 and 15 with PBS, 0.25 mg/kg PNU-MES-ADC or 30 μg/kg free PNU159682 (equivalent toxin amount as in the 0.25 mg/kg PNU-MES-ADC dose), or on day 1 once with 0.75 mg/kg PNU-MES-ADC. Mice were followed for over 50 days for tumor response and health. As shown in FIG. 5 at day 49, a single dose of MES-ADC at 0.75 mg/kg in a cleavable format was sufficient to significantly reduce established tumor growth (P≤0.0061) and maintain a durable response similar to 3 weekly doses of PNU-MES-ADC at 0.25 mg/kg in contrast to PBS or free PNU159682 (PN-free) treated mice, demonstrating the efficacy of this format and composition for treating mesothelin-expressing tumors with or without CA125 expression. Drug treatments were well tolerated across all groups.

These data support the teaching that composition of matter containing the PNU-MES-ADC and SN38-MES-ADCs in cleavable formats are able to kill tumors effectively with immunosuppressed and immune-proficient tumor microenvironments. The finding that other payloads such as MMAE microtubule inhibitor or linkers such as the non-enzymatic cleavable MC-EDA are less effective teaches that the generic use of a tumor targeting ADC cannot simply overcome tumor immunosuppression, but rather: 1) active screening for antibodies not bound by immunosuppressive factors such as CA125, along with 2) screening for optimal payload cytotoxicity, and 3) screening for optimal linkers are required for developing a potent and effective ADC agent capable of treating immunosuppressed and immunoproficient tumors as described in the inventions taught here.

### Example 3—Generation of Anti-Mesothelin Bispecific Antibodies (MES-BSP) Capable of Killing Immunosuppressed and Immunoproficient Mesothelin-Expressing Cancer Cells

The use of antibodies to target tumor cells has typically involved the blockade of cytokine binding to a cytokine receptor to suppress tumor cell growth and/or the use of humoral mediated immune killing via ADCC, CDC and/or immune-effector cellular opsonization. As described above, the tumor produced CA125 protein as well as others tumor produced proteins (the latter are N.C.N, J.B.K. L.G. personal observations) have the ability to suppress humoral-mediated antibody killing of target cells. The use of antibodies that are naturally refractory to binding of immunosuppressive proteins enables their tumor cell killing even in the presence of such proteins. As described in Example 1, the MES-1 antibody was identified by screening anti-mesothelin antibodies for their ability to avoid CA125 binding. To enhance the immune-mediated killing of MES-1, we engineered it to potentially be able to use immune-mediated killing via recruitment of CD3⁺ cytotoxic T-cells to the target cell's proximal surface and subsequent T-cell activation, a feature critical for BSP antibody tumor cell killing (Staerz U D, et al. Nature 314; 628-631, 1985). The inventors teach here of the use of a CA125 non-binding, anti-mesothelin antibody genetically fused a second antibody that can bind to a cell surface antigen expressed on T-lymphocytes that leads to improved immune-mediated tumor cell killing irrespective of the tumor microenvironment immune status. As an example, we demonstrate the use of a single chain antibody that can bind to the CD3 antigen on T-cells fused to the MES-1 antibody. As discussed in Example 2, Nicolaides et. al. (Cancer Biol Ther 19:622-630, 2018) have previously shown that CA125 binding to anti-mesothelin antibody amatuximab results in suppressed humoral immune function. To determine the maximal efficacy that a MES-1 antibody in BSP format may have against tumors with immunoproficient and immunosuppressive microenvironments, we considered several MES-BSP formats, linking the anti-CD3 single chain antibody (SEQ ID NO: 6) to MES-1 light (SEQ ID NO: 1) or heavy chain (SEQ ID NO: 2) and proceeded with a fusion of CD3 single chain to the N-terminus of the MES-1 light chain using an amino acid linker GGGS as shown in SEQ ID NO: 3 and the canonical MES-1 heavy chain (SEQ ID NO: 2). We then generated recombinant MES-BSP antibody as described below and tested its activity against cancer cell lines that are immunoproficient and immunosuppressed by the CA125 protein.

To produce high quality MES-BSP, we engineered recombinant Chinese hamster ovarian (CHO) cells to express the MES-BSP for large scale production. The CD3 single chain antibody was cloned upstream of the mature N-terminal domain of the MES-1 light chain via a genetic linker encoding the amino acids GGGGS as shown in SEQ ID NO: 3. The MES-1 light chain-CD3 single chain fused cDNA and the MES-1 heavy chain cDNA were cloned into the pXC vector that has two CMV driven expression cassettes and the glutamine synthase (GS) gene cassette, referred here as pNAV0071. To generate stable recombinant fusion protein production cell lines, CHOK1SV-GSKO cells containing a knocked out endogenous GS gene were cultured at 6×10⁵ cell/mL in CD-CHO (Irving Scientific) plus 6 mM L-Glutamine overnight at 37° C. in 5% CO₂. The next day, 2.0×10⁷ cells were resuspended with 20 μg expression plasmid pNAV0071 in a total volume of 700 μL plain CD-CHO, then transferred to a 0.4 cm electroporation cuvette and electroporated at 300 V/900 μF using the BioRad GenePulser II. Cells were immediately transferred to a flask containing 30 mL of warm CD-CHO plus 6 mM L-Glutamine and incubated overnight at 37° C. in 5% CO₂ in a shaking platform incubator. The following day, cells were harvested and resuspended in 30 mL CD-CHO/SP4 containing 50 μM MSX for selection. Next, the selected pool was subcloned by limiting dilution and conditioned media were tested for recombinant MES-BSP antibody production from established clones via ELISA using an anti-human Fc-HRP as probe. Productive subclones (producing greater than 0.5 mg/mL) were expanded and analyzed for antibody production and quality (target antigen binding and protein homogeneity). The best quality clone was then expanded and MES-BSP antibody was purified from culture medium using Protein A column affinity chromatography and dialysis in PBS buffer. The MES-BSP antibody was then quantified and analyzed for homogeneity via SDS-PAGE analysis and antigen binding via ELISA. High quality preps were then tested for efficacy against various tumor cell lines.

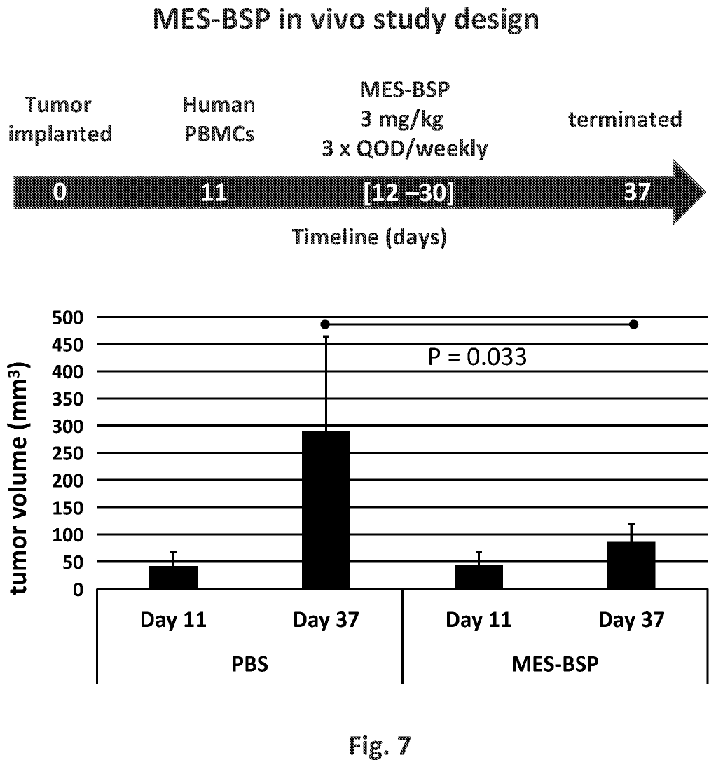

MES-BSP was first tested for humoral immune killing via ADCC, comparing it to the parental MES-1 antibody and the humoral immunosuppressed meso-Ab-4 antibody (Ab-4, lane 4, FIG. 1) in the presence of the mesothelin-expressing, immunosuppressed OVCAR3 tumor cell line that naturally over-expresses the CA125 protein and human PBMCs. As shown in FIG. 6A, MES-BSP had significantly higher killing against the CA125 producing OVCAR-3 cell line as compared to MES-1 or meso-Ab-4, the latter of which showed no killing activity. This appeared to be CA125 driven as ADCC activity of meso-Ab-4 was similar to that of MES-1 against the OVCAR3CA125 knockdown OVCAR-KO cell line (FIG. 6B). In this assay, the Jurkat-CD16a ADCC reporter cell line was used to monitor ADCC activity via a luciferase readout following the manufacturer's instructions (Promega Corp). A similar line was previously published and shown to have significantly enhanced antibody-mediated humoral response by CA125 affected antibodies in contrast to parental OVCAR cells (Kline J B, et al. OncoTarget 8:52045-52060, 2017; Nicolaides N C, et al. Cancer Biol Ther 13:1-22, 2018). Finally, to demonstrate in vivo efficacy of MES-BSP against mesothelin-expressing, CA125 positive tumor cells, we employed a humanized PBMC mouse model, whereby athymic nude mice were implanted with a mesothelioma-derived tumor cells expressing both mesothelin and CA125. Upon establishment of the tumors, the mice were implanted with human peripheral blood mononuclear cells (PBMCs) followed by MES-BSP or control treatment. As shown in FIG. 7, MES-BSP was able to statistically suppress tumor growth, confirming its utility in treating mesothelin-expressing cells in CA125 immunosuppressed tumor microenvironments. Here we teach that CA125 may have a significant impact on the efficacy of antibodies using immune-mediated killing in which CA125 is able to bind. Moreover, we provide compositions by which anti-mesothelin BSP antibodies can effectively kill immunosuppressed tumor cells.

TABLE 2
SEQUENCE IDENTIFICATION (all sequences N to C terminal)

Underline denotes antibody CDRs in the anti-mesothelin directed antibody
Underline italics denotes CDRs in the anti-CD3 directed antibody
BOLD represents light or heavy chain constant regions
Bold italics represents spacers between the CD3 single light and heavy chain and between the
CD3 single chain and anti-mesothelin light chain fusion
SEQ ID NO: 1 (MES light chain)
DIQMTQSPSSLSASVGDRVTITCQASQRISSYLSWYQQKPGKVPKLLIYGASTLASGVPSRFSGSGSGTDFTLTISS
LQPEDVATYYCQSYAYFDSNNWHAFGGGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
SEQ IN NO: 2 (MES heavy chain)
EVQLVESGGGLVQPGGSLRLSCAASGFDLGFYFYACWVRQAPGKGLEWVSCIYTAGSGSTYYASWAKGRFTISRDNS
KNTLYLQMNSLRAEDTAVYYCARSTANTRSTYYLNLWGQGTLVTVSSASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSCDKTH
TCPPCPAPELLGGPSVFLFPPKPKDTLMISRTPEVTCVVVDVSHEDPEVKENWYVDGVEVHNAKTKPREEQYNSTYR
VVSVLTVLHQDWLNGKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSLSLSPG
SEQ IN NO: 3 (MES-BSP: single chain anti-CD3 fused to MES light chain)
QVQLVESGGGVVQPGRSLRLSCAASGYTFTRYTMHWVRQAPGKCLEWIGYINPSRGYTNYNQKVKGRFTISRDNSKN
TAYLQMNSLRAEDTAVYYCARYYDDHYCLDYWGQGTLVTVSS [symbol] DIQMTQSPSSLSASV
GDRVTITCSASSSVSYMNWYQQKPGKAPKLLIYDTSKLASGVPSRFSGSGSGTDYTFTISSLQPEDIATYYCQQWSS
NPFTFGCGTKLEIK [symbol] DIQMTQSPSSLSASVGDRVTITCQASQRISSYLSWYQQKPGKVPKLLIYGASTLASGV
PSRFSGSGSGTDFTLTISSLQPEDVATYYCQSYAYFDSNNWHAFGGGTKVEIKRTVAAPSVFIFPPSDEQLKSGTAS
VVCLLNNFYPREAKVQWKVDNALQSGNSQESVTEQDSKDSTYSLSSTLTLSKADYEKHKVYACEVTHQGLSSPVTKS
FNRGEC
The anti-CD3 VH is before first GGGGSₓ linker and the anti-CD3 VL is between
first and second linkers, while the anti-mesothelin VL is after second
linker.
SEQ ID NO: 4 (MES-BSP: single chain anti-CD3 fused to MES light chain) with
modified leader sequence underlined
atgtctgtgcctacccaggtgctgggactgctgctgctgtggctgacagacgcccgctgtcaagtgcagttggtgga
atcagggggaggagtcgtgcagccgggaagatcattgagactgtcgtgcgcggcgtccggttacaccttcacccggt
atactatgcactgggtgcgccaggcccctggcaaatgcctggagtggatcggttacattaacccgagcagggggtac
accaactacaaccagaaggtcaagggccgcttcaccatctcccgggataactccaagaacaccgcatacctccaaat
gaactccctgcgggccgaagatacggccgtgtactactgtgcccggtactacgacgaccattactgccttgactact
ggggccagggcactctggtgactgtgtccagcgggggcggtggaagcggggggggaggctccggaggaggcggatcg
ggtggcggcggcagcgacatccaaatgacccagtccccgtcctcactttccgcatccgtcggcgatcgcgtgaccat
tacttgttccgcgtcgtcgtccgtgagctacatgaactggtatcagcagaagccaggaaaggccccgaaactgctga
tctacgacacttccaagctggcttctggagtgcccagcagattcagcggatcagggtccggtaccgactacaccttc
accatttcgtccctgcaacccgaagatatcgccacctactactgccagcagtggtcgagcaacccttttacgttcgg
ctgtggcaccaagctcgagatcaaaggtggcggcggtagcgacatccagatgacacaatctccttcatctctcagtg
cttccgtaggagatagagttactataacctgtcaagcatctcaaaggatctcttcctatctcagttggtatcaacag
aaaccgggaaaagtgcccaaacttcttatctacggtgctagtacacttgcttccggggtcccctcaaggttcagcgg
cagcggttctggaacagactttaccctgacgatctcaagtctccagccagaagacgtggctacatactactgccagt
cttacgcatacttcgatagcaataactggcacgccttcggtggcggaaccaaagttgaaataaaacgaactgtggct
gcaccatctgtcttcatcttcccgccatctgatgagcagttgaaatctggaactgcctctgttgtgtgcctgctgaa
taacttctatcccagagaggccaaagtacagtggaaggtggataacgccctccaatcgggtaactcccaggagagtg
tcacagagcaggacagcaaggacagcacctacagcctcagcagcaccctgacgctgagcaaagcagactacgagaaa
cacaaagtctacgcctgcgaagtcacccatcagggcctgagctcgcccgtcacaaagagcttcaacaggggagagtg
t
SEQ ID NO: 5 (MES heavy chain) with modified leader sequence underlined
atggaatggagctgggtgttcctgttctttctgtccgtgaccacaggcgtgcattctgaagtgcaactcgtggagt
caggcgggggtctggttcagccgggcggcagtcttcggcttagttgtgccgcaagcggctttgacctcgggtt
ttatttctacgcctgttgggtaaggcaggcacctggaaagggtctggaatgggtctcttgcatatatacggca
ggtagcggctccacgtattacgcaagttgggccaagggccggttcacaatatctagggacaattccaaaaata
ccctgtacctgcaaatgaacagtctcagggctgaagacactgctgtctactattgcgctcgctcaacggctaa
tacccggtccacttattacttgaacctctggggtcagggaactttggtaacagtatcatccgcatccaccaag
ggcccatcggtcttccccctggcaccctcctccaagagcacctctgggggcacagcggccctgggctgcctgg
tcaaggactacttccccgaaccggtgacggtgtcgtggaactcaggcgccctgaccagcggcgtgcacacctt
cccggctgtcctacagtcctcaggactctactccctcagcagcgtggtgaccgtgccctccagcagcttgggc
acccagacctacatctgcaacgtgaatcacaagcccagcaacaccaaggtggacaagaaagttgagcccaaat
cttgtgacaaaactcacacatgcccaccgtgcccagcacctgaactcctggggggaccgtcagtcttcctctt
ccccccaaaacccaaggacaccctcatgatctcccggacccctgaggtcacatgcgtggtggtggacgtgagc
cacgaagaccctgaggtcaagttcaactggtacgtggacggcgtggaggtgcataatgccaagacaaagccgc
gggaggagcagtacaacagcacgtaccgtgtggtcagcgtcctcaccgtcctgcaccaggactggctgaatgg
caaggagtacaagtgcaaggtctccaacaaagccctcccagcccccatcgagaaaaccatctccaaagccaaa
gggcagccccgagaaccacaggtgtacaccctgcccccatcccgggatgagctgaccaagaaccaggtcagcc
tgacctgcctggtcaaaggcttctatcccagcgacatcgccgtggagtgggagagcaatgggcagccggagaa
caactacaagaccacgcctcccgtgctggactccgacggctccttcttcttatattcaaagctcaccgtggac
aagagcaggtggcagcaggggaacgtcttctcatgctccgtgatgcatgaggctctgcacaaccactacacgc
agaagagcctctccctgtctccgggt
SEQ ID NO: 6 [anti-CD3 HC and LC single chain]
QVQLVESGGGVVQPGRSLRLSCAASGYTFTRYTMHWVRQAPGKCLEWIGYINPSRGYTNYNQKVKGRFTISRDNSKN
TAYLQMNSLRAEDTAVYYCARYYDDHYCLDYWGQGTLVTVSS [symbol] DIQMTQSPSSLSASV
GDRVTITCSASSSVSYMNWYQQKPGKAPKLLIYDTSKLASGVPSRFSGSGSGTDYTFTISSLQPEDIATYYCQQWSS
NPFTFGCGTKLEIK
SEQ ID NO: 7
QASQRISSYLS
SEQ ID NO: 8
GASTLAS
SEQ ID NO: 9
QSYAYFDSNNWHA
SEQ ID NO: 10
GFDLGFYFYAC
SEQ ID NO: 11
CIYTAGSGSTYYASWAKG
SEQ ID NO: 12
STANTRSTYYLNL
SEQ ID NO: 13
GYTFTRYTMH
SEQ ID NO: 14
WIGYINPSRGYTNYNQKVKG
SEQ ID NO: 15
ARYYDDHYCLDY
SEQ ID NO: 16
SASSSVSYMNWY
SEQ ID NO: 17
LLIYDTSKLAS
SEQ ID NO: 18
QQWSSNPFT
SEQ ID NO: 19 [MES light chain cDNA] with modified leader sequence underlined
atgtctgtgcctacccaggtgctgggactgctgctgctgtggctgacagacgcccgctgtgacatacagatgactca
gtccccttcaagcctcagtgcatcagtaggtgaccgggttaccattacctgccaggccagtcaacgaatatcatcct
acctctcctggtaccagcagaagccggggaaggtgcctaagctcttgatctacggcgccagtacgcttgcaagcggg
gtcccatcacggttctccggtagtggctctggaacagattttacgctcacgatttccagccttcaaccggaggatgt
tgcgacttactactgtcaatcctatgcgtatttcgattccaataactggcacgcattcggtgggggaaccaaagtgg
agattaaacgaactgtggctgcaccatctgtcttcatcttcccgccatctgatgagcagttgaaatctggaactgcc
tctgttgtgtgcctgctgaataacttctatcccagagaggccaaagtacagtggaaggtggataacgccctccaatc
gggtaactcccaggagagtgtcacagagcaggacagcaaggacagcacctacagcctcagcagcaccctgacgctga
gcaaagcagactacgagaaacacaaagtctacgcctgcgaagtcacccatcagggcctgagctcgcccgtcacaaag
agcttcaacaggggagagtgt

1. An antibody-drug conjugate (ADC) comprising an anti-mesothelin antibody comprising complementary determining regions (CDRs) with amino acids as shown in SEQ ID NOs: 7-12, and a topoisomerase inhibitor.

2. The ADC of claim 1 wherein the antibody comprises the amino acid sequences of SEQ ID NO:1 and SEQ ID NO:2.

3. The ADC of claim 1 wherein the antibody is covalently linked to the topoisomerase inhibitor.

4. The ADC of claim 1 wherein the antibody is linked to a topoisomerase inhibitor through a cleavable linker.

5. The ADC of claim 4 which is encapsulated in a liposome.

6. The ADC of claim 3 wherein the topoisomerase inhibitor is (2S,4S)-2,5,12-trihydroxy-7-methoxy-4-{[(1S,3R,4aS,9S,9aR,10aS)-9-methoxy-1-methyloctahydro-1H-pyrano[4′,3′:4,5][1,3]oxazolo[2,3-c][1,4]oxazin-3-yl]oxy}-6,11-dioxo-1,2,3,4,6,11-hexahydrotetracene-2-carboxylic acid (PNU159682).

7. The ADC of claim 6 wherein PNU159682 is covalently linked to the anti-mesothelin antibody via linker maleimidocaproyl-valine-citrulline-p-aminobenzoyloxycarbonyl (MA-PEG4-VC-PAB-DMAE).

8. The ADC of claim 7 wherein the linker is linked to cysteines in the anti-mesothelin antibody.

9. The ADC of claim 7 wherein the antibody comprises the amino acid sequences of SEQ ID NO:1 and SEQ ID NO: 2.

10. The ADC of claim 9 wherein the DAR of the ADC is between 2 and 6, inclusive.

11. The ADC of claim 3 wherein the topoisomerase inhibitor is (2S,3S,4S,5R,6S)-6-[[(19S)-10,19-diethyl-19-hydroxy-14,18-dioxo-17-oxa-3,13-diazapentacyclo[11.8.0.02,11.04,9.015,20]henicosa-1(21),2,4(9),5,7,10,15(20)-heptaen-7-yl]oxy]-3,4,5-trihydroxyoxane-2-carboxylic acid (SN38).

12. The ADC of claim 11 wherein SN38 is covalently linked to the anti-mesothelin antibody via linker methyl (2S,3S,4S,5R,6S)-3,4,5-triacetyloxy-6-[2-amino-4-(hydroxymethyl)phenoxy]oxane-2-carboxylate (MAC-glucuronide).

13. The ADC of claim 11 wherein SN38 is covalently linked to the anti-mesothelin antibody via linker PEG8 triazole-PABC-peptide-MC.

14. The ADC of claim 11 wherein SN38 is covalently linked to cysteines in the anti-mesothelin antibody.

15. The ADC of claim 11 wherein the anti-mesothelin antibody comprises amino acid sequences SEQ ID NO:1 and SEQ ID NO: 2.

16. The ADC of claim 15 wherein the ADC has a DAR of between 2 and 6, inclusive.

17. A method of treating a cancer patient who has a mesothelin-expressing tumor, comprising:
administering to the patient an antibody-drug conjugate (ADC) comprising an anti-mesothelin antibody comprising amino acids as shown in SEQ ID NOs: 7-12, and a topoisomerase inhibitor.

18. The method of claim 17 wherein the cancer is selected from the group consisting of mesothelioma, breast, lung, colorectal, gastro-intestinal, endometrial, cholangial and pancreatic cancers.

19. The method of claim 17 further comprising the step of:
detecting in the patient the presence of a mesothelin epitope by contacting a body sample from the patient with an antibody which comprises amino acid sequences of SEQ ID NOs: 7-12.

20. The method of claim 17 wherein the patient has a high level of CA125 relative to a population of healthy humans.

21. The method of claim 17 wherein a plurality of patients are treated by administering the ADC, wherein the plurality of patients comprises at least one patient that has a high level of CA125 and at least one patient that has a normal level of CA125 relative to a population of healthy humans.

22. A bispecific antibody (BSP) comprising a mesothelin-binding portion comprising amino acid sequences SEQ ID NOs: 7-12, and a cell surface antigen CD3-binding portion.

23. The bispecific antibody of claim 22 wherein the cell surface antigen CD3-binding portion comprises CDR amino acid sequences SEQ ID NOs: 13-18.

24. The bispecific antibody of claim 22 wherein the bispecific antibody comprises a light chain of an anti-mesothelin antibody comprising the amino acid sequence of SEQ ID NO: 1 fused to a single chain antibody that recognizes human cell surface antigen CD3 comprising the amino acid sequence of SEQ ID NO: 6.

25. The bispecific antibody of claim 22 wherein the bispecific antibody comprises a light chain of an anti-mesothelin antibody comprising the amino acid sequence of SEQ ID NO: land said light chain is linked to the CD3 single chain antibody by a spacer unit comprising one or more units of amino acids GGGGS (SEQ ID NO: 20).

26. A nucleic acid vector which encodes the bispecific antibody of claim 22.

27. The nucleic acid vector of claim 26 comprising the nucleic acid sequences of SEQ ID NO: 4 and SEQ ID NO: 5.

28. A stable cell line comprising one or more nucleic acids encoding the bispecific antibody of claim 22.

29. A stable cell line according to claim 28 wherein the one or more nucleic acids comprise the nucleic acid sequences of SEQ ID NO: 4 and SEQ ID NO: 5.

30. A method to treat a mesothelin-expressing cancer in a patient, comprising:
administering to the patient the bispecific antibody of claim 22, thereby treating the mesothelin-expressing cancer.

31. The method of claim 30 wherein the mesothelin-expressing cancer is selected from the group consisting of mesothelioma, breast, lung, colorectal, gastro-intestinal, endometrial, cholangial and pancreatic cancers.

32. The method of claim 30 further comprising the step of:
testing the patient by contacting a body sample of the patient with an antibody which comprises complementarity determining regions (CDRs) comprising the amino acid sequences of SEQ ID NOs: 7-12, thereby detecting a mesothelin epitope in the cancer in the patient.

33. The method of claim 30 wherein a plurality of patients are treated by administering the bispecific antibody, wherein the plurality of patients comprises at least one patient that has a high level of CA125 relative to a population of healthy humans and at least one patient that has a normal level of CA125.

34. The method of claim 30 wherein the patient has a high level of CA125 relative to a population of healthy humans.


SEQUENCE LISTING (20 sequences)

SEQ ID NO: 1 - protein, 218 aa, Oryctolagus cuniculus
DIQMTQSPSSLSASVGDRVTITCQASQRISSYLSWYQQKPGKVPKLLIYGASTLASGVPSRFSGSGSGTDFTLTISSLQPEDVATYYCQSYAYFDSNNWHAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 453 aa, Oryctolagus cuniculus
EVQLVESGGGLVQPGGSLRLSCAASGFDLGFYFYACWVRQAPGKGLEWVSCIYTAGSGSTYYASWAKGRFTISRDNSKNTLYLQMNSLRAEDTAVYYCARSTANTRSTYYLNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 468 aa, Artificial Sequence
  modified antibody sequences by fusion
QVQLVESGGGVVQPGRSLRLSCAASGYTFTRYTMHWVRQAPGKCLEWIGYINPSRGYTNYNQKVKGRFTISRDNSKNTAYLQMNSLRAEDTAVYYCARYYDDHYCLDYWGQGTLVTVSSGGGGSGGGGSGGGGSGGGGSDIQMTQSPSSLSASVGDRVTITCSASSSVSYMNWYQQKPGKAPKLLIYDTSKLASGVPSRFSGSGSGTDYTFTISSLQPEDIATYYCQQWSSNPFTFGCGTKLEIKGGGGSDIQMTQSPSSLSASVGDRVTITCQASQRISSYLSWYQQKPGKVPKLLIYGASTLASGVPSRFSGSGSGTDFTLTISSLQPEDVATYYCQSYAYFDSNNWHAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - DNA, 1464 bp, Artificial Sequence
  modified antibody sequences by fusion
atgtctgtgcctacccaggtgctgggactgctgctgctgtggctgacagacgcccgctgtcaagtgcagttggtggaatcagggggaggagtcgtgcagccgggaagatcattgagactgtcgtgcgcggcgtccggttacaccttcacccggtatactatgcactgggtgcgccaggcccctggcaaatgcctggagtggatcggttacattaacccgagcagggggtacaccaactacaaccagaaggtcaagggccgcttcaccatctcccgggataactccaagaacaccgcatacctccaaatgaactccctgcgggccgaagatacggccgtgtactactgtgcccggtactacgacgaccattactgccttgactactggggccagggcactctggtgactgtgtccagcgggggcggtggaagcggggggggaggctccggaggaggcggatcgggtggcggcggcagcgacatccaaatgacccagtccccgtcctcactttccgcatccgtcggcgatcgcgtgaccattacttgttccgcgtcgtcgtccgtgagctacatgaactggtatcagcagaagccaggaaaggccccgaaactgctgatctacgacacttccaagctggcttctggagtgcccagcagattcagcggatcagggtccggtaccgactacaccttcaccatttcgtccctgcaacccgaagatatcgccacctactactgccagcagtggtcgagcaacccttttacgttcggctgtggcaccaagctcgagatcaaaggtggcggcggtagcgacatccagatgacacaatctccttcatctctcagtgcttccgtaggagatagagttactataacctgtcaagcatctcaaaggatctcttcctatctcagttggtatcaacagaaaccgggaaaagtgcccaaacttcttatctacggtgctagtacacttgcttccggggtcccctcaaggttcagcggcagcggttctggaacagactttaccctgacgatctcaagtctccagccagaagacgtggctacatactactgccagtcttacgcatacttcgatagcaataactggcacgccttcggtggcggaaccaaagttgaaataaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 5 - DNA, 1416 bp, Artificial Sequence
  modified antibody sequences with leader sequence
atggaatggagctgggtgttcctgttctttctgtccgtgaccacaggcgtgcattctgaagtgcaactcgtggagtcaggcgggggtctggttcagccgggcggcagtcttcggcttagttgtgccgcaagcggctttgacctcgggttttatttctacgcctgttgggtaaggcaggcacctggaaagggtctggaatgggtctcttgcatatatacggcaggtagcggctccacgtattacgcaagttgggccaagggccggttcacaatatctagggacaattccaaaaataccctgtacctgcaaatgaacagtctcagggctgaagacactgctgtctactattgcgctcgctcaacggctaatacccggtccacttattacttgaacctctggggtcagggaactttggtaacagtatcatccgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctccgggt

SEQ ID NO: 6 - protein, 245 aa, Artificial Sequence
  modified antibody sequences by fusion
QVQLVESGGGVVQPGRSLRLSCAASGYTFTRYTMHWVRQAPGKCLEWIGYINPSRGYTNYNQKVKGRFTISRDNSKNTAYLQMNSLRAEDTAVYYCARYYDDHYCLDYWGQGTLVTVSSGGGGSGGGGSGGGGSGGGGSDIQMTQSPSSLSASVGDRVTITCSASSSVSYMNWYQQKPGKAPKLLIYDTSKLASGVPSRFSGSGSGTDYTFTISSLQPEDIATYYCQQWSSNPFTFGCGTKLEIK

SEQ ID NO: 7 - protein, 11 aa, Artificial Sequence
  linker
QASQRISSYLS

SEQ ID NO: 8 - protein, 7 aa, Artificial Sequence
  llinker
GASTLAS

SEQ ID NO: 9 - protein, 13 aa, Artificial Sequence
  linker
QSYAYFDSNNWHA

SEQ ID NO: 10 - protein, 11 aa, Artificial Sequence
  linker
GFDLGFYFYAC

SEQ ID NO: 11 - protein, 18 aa, Artificial Sequence
  linker
CIYTAGSGSTYYASWAKG

SEQ ID NO: 12 - protein, 13 aa, Artificial Sequence
  linker
STANTRSTYYLNL

SEQ ID NO: 13 - protein, 10 aa, Artificial Sequence
  linker
GYTFTRYTMH

SEQ ID NO: 14 - protein, 20 aa, Artificial Sequence
  linker
WIGYINPSRGYTNYNQKVKG

SEQ ID NO: 15 - protein, 12 aa, Artificial Sequence
  linker
ARYYDDHYCLDY

SEQ ID NO: 16 - protein, 12 aa, Artificial Sequence
  linker
SASSSVSYMNWY

SEQ ID NO: 17 - protein, 11 aa, Artificial Sequence
  linker
LLIYDTSKLAS

SEQ ID NO: 18 - protein, 9 aa, Artificial Sequence
  linker
QQWSSNPFT

SEQ ID NO: 19 - DNA, 720 bp, Artificial Sequence
  modified antibody sequences with modifified leader sequence
atgtctgtgcctacccaggtgctgggactgctgctgctgtggctgacagacgcccgctgtgacatacagatgactcagtccccttcaagcctcagtgcatcagtaggtgaccgggttaccattacctgccaggccagtcaacgaatatcatcctacctctcctggtaccagcagaagccggggaaggtgcctaagctcttgatctacggcgccagtacgcttgcaagcggggtcccatcacggttctccggtagtggctctggaacagattttacgctcacgatttccagccttcaaccggaggatgttgcgacttactactgtcaatcctatgcgtatttcgattccaataactggcacgcattcggtgggggaaccaaagtggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtctgcac

SEQ ID NO: 20 - protein, 5 aa, Artificial Sequence
  linker
GGGGS